(19) United States
(12) Patent Application Publication
(10) Pub. No.: US 2025/0029372 A1
(43) Pub. Date: Jan. 23, 2025

(54) SYSTEMS, METHODS, AND APPARATUSES FOR IMPLEMENTING STEPWISE INCREMENTAL PRE-TRAINING FOR INTEGRATING DISCRIMINATIVE, RESTORATIVE, AND ADVERSARIAL LEARNING INTO AN AI MODEL

(71) Applicant: Arizona Board of Regents on behalf of Arizona State University, Scottsdale, AZ (US)

(72) Inventors: Zuwei Guo, Phoenix, AZ (US); Nahid Ul Islam, Mesa, AZ (US); Jianming Liang, Scottsdale, AZ (US)

(21) Appl. No.: 18/776,010

(22) Filed: Jul. 17, 2024

Related U.S. Application Data
(60) Provisional application No. 63/514,037, filed on Jul. 17, 2023.

(51) Int. Cl. G06V 10/774; G06N 3/0455; G06N 3/088; G06N 3/094; G06V 10/82; G16H 30/40

(52) U.S. Cl. CPC G06V 10/774; G06N 3/0455; G06N 3/088; G06N 3/094; G06V 10/82; G16H 30/40; G06V 2201/03

(57) ABSTRACT

A stepwise incremental pre-training for integrating discriminative, restorative, and adversarial learning in an AI model. Exemplary systems include means for receiving a training dataset for training a unified AI model. The unified AI model applies each of discriminative, restorative, and adversarial learning operations through three transferable components: a discriminative encoder, a restorative decoder, and an adversarial encoder. Stepwise incremental pre-training operations train the unified AI model, including pre-training the discriminative encoder via discriminative learning and attaching the trained discriminative encoder with the restorative decoder to form a skip-connected encoder-decoder, pre-training the skip-connected encoder-decoder via joint discriminative and restorative learning, and associating the pre-trained skip-connected encoder-decoder with the adversarial encoder. Training of the AI model is finalized by performing full discriminative, restorative, and adversarial learning on the training dataset using the unified AI model.

### CLAIM OF PRIORITY

This application claims the benefit of U.S. Provisional Patent Application No. 63/514,037, filed Jul. 17, 2023, entitled “SYSTEMS, METHODS, AND APPARATUSES FOR IMPLEMENTING STEPWISE INCREMENTAL PRE-TRAINING FOR INTEGRATING DISCRIMINATIVE, RESTORATIVE, AND ADVERSARIAL LEARNING INTO AN AI MODEL”, the disclosure of which is incorporated by reference herein in its entirety.

### GOVERNMENT RIGHTS AND GOVERNMENT AGENCY SUPPORT NOTICE

This invention was made with government support under R01 HL128785 awarded by the National Institutes of Health. The government has certain rights in the invention.

### COPYRIGHT NOTICE

A portion of this document contains material which is subject to copyright protection. The copyright owner has no objection to the facsimile reproduction by anyone of the document as it appears in the Patent and Trademark Office patent records, but otherwise reserves all copyright rights whatsoever.

### TECHNICAL FIELD

Embodiments of the invention relate generally to the field of medical imaging and analysis using convolutional neural networks and transformers for the classification and annotation of medical images, and more particularly, to systems, methods, and apparatuses for implementing stepwise incremental pre-training for integrating discriminative, restorative, and adversarial learning into a single Artificial Intelligence (AI) model, in the context of medical image analysis.

### BACKGROUND

Machine learning models have various applications to automatically process inputs and produce outputs considering situational factors and learned information to improve output quality. One area where machine learning models, and neural networks in particular, provide high utility is in the field of processing medical images.

In the context of machine learning and deep learning specifically, a Convolutional Neural Network (CNN, or ConvNet) is a class of deep neural networks, very often applied to analyzing visual imagery. Convolutional Neural Networks are regularized versions of multilayer perceptrons. Multilayer perceptrons are fully connected networks, such that each neuron in one layer is connected to all neurons in the next layer, a characteristic which often leads to a problem of overfitting of the data and the need for model regularization. Convolutional Neural Networks also seek to apply model regularization, but with a distinct approach. Specifically, CNNs take advantage of the hierarchical pattern in data and assemble more complex patterns using smaller and simpler patterns. Consequently, on the scale of connectedness and complexity, CNNs are on the lower extreme.

Unfortunately, prior known techniques regardless of using unsupervised or supervised learning modes fail to yield trained AI models capable of providing discriminative, restorative, and adversarial learning components with sufficient unification to provide state of the art results.

What is needed is an improved technique for the integrating and unifying the benefits of discriminative, restorative, and adversarial learning into a single trained AI model variant capable of yielding equal or greater performance when compared with known alternatives.

The present state of the art may therefore benefit from the systems, methods, and apparatuses for implementing stepwise incremental pre-training for integrating discriminative, restorative, and adversarial learning into a single AI model, as is described herein.

### BRIEF DESCRIPTION OF THE DRAWINGS

Embodiments are illustrated by way of example, and not by way of limitation, and can be more fully understood with reference to the following detailed description when considered in connection with the figures in which:

FIG. 1 depicts an exemplary “United” model which consists of three distinct components: a discriminative encoder E, a restorative decoder D, and an adversary encoder A, where the discriminative encoder and the restorative decoder are skip connected, forming an encoder-decoder (E, D), according to embodiments of the invention.

FIGS. 2A, 2B, 2C, 2D, 2E, 2F, 2G, 2H and 2I depict a novel redesign of nine Self-Supervised Learning (SSL) methods, including (a) Jigsaw, (b) Rubik's Cube, (c) Deep Clustering, (d) Rotation, (e) TransVW, (f) MoCo, (g) BYOL, (h) Swin UNTRE, and (i) PCRL within the exemplary United framework.

FIG. 3A presents Table 1, depicting that, when training a United model continually component-by-component, the stepwise incremental pre-training may choose to train the components in different sequences, leading to various pre-training strategies.

FIG. 3B presents Table 2, showing how the strategy D(D+R)(D+R+A) always outperforms Strategy D+R+A on all nine target tasks.

FIG. 3C presents Table 3, showing how discriminative learning enhances discriminative encoders for classification and segmentation tasks.

FIG. 3D presents Table 4, showing how incremental restorative pre-training combined with continual discriminative learning (i.e., Strategy D(D+R)) enhances discriminative encoders for classification tasks.

FIG. 3E presents Table 5, showing how incremental restorative pre-training combined with continual discriminative learning (i.e., Strategy D(D+R)) directly boost target segmentation tasks.

FIGS. 4A, 4B, and 4C show how the incremental adversarial training in Strategy D(D+)(D+R+A) strengthens learned representation.

FIGS. 4D, 4E, and 4F show how the stepwise incremental pre-training D(D+R)(D+R+A) helps reduce annotation costs.

FIGS. 5A, 5B, 5C, 5D, 5E, and 5F show the different variants tested for the stepwise incremental pre-training scheme R(R+D) and D(D+R) with components D and R.

FIG. 6 presents Table 6, shows how the final stepwise incremental pre-training (Step D(D+R)(D+R+A)) generates sharper and more realistic images for restoration tasks.

FIGS. 7A, 7B, and 7C present Tables 7A, 7B, and 7C, showing a comparison of different training strategies.

FIG. 8 presents Table 8, showing how adversarial encoders have learned weak representation, and that they are not suitable for transfer learning.

FIG. 9 sets forth Equations 1 through 5, which are referenced throughout the disclosure.

### DETAILED DESCRIPTION

Described herein are systems, methods, and apparatuses for implementing stepwise incremental pre-training for integrating discriminative, restorative, and adversarial learning into a single AI model, in the context of medical image analysis.

A United framework implementation, according to embodiments of the invention, and which is described in greater detail below, integrates three Self-Supervised Learning (SSL) ingredients (discriminative, restorative, and adversarial learning), enabling collaborative learning among the three learning ingredients and yielding three transferable components: a discriminative encoder, a restorative decoder, and an adversary encoder.

Nine prominent self-supervised methodologies are redesigned, including Rotation, Jigsaw, Rubik's Cube, Deep Clustering, TransVW, MoCo, BYOL, Swin UNTRE and PCRL, each being augmented with its missing components in a United framework for 3D medical imaging.

However, such a United framework increases model complexity, making pre-training difficult in 3D imaging applications. To overcome this difficulty, stepwise incremental pre-training is further developed and configured, resulting in a strategy that unifies the pre-training, in which a discriminative encoder is first trained via discriminative learning, the pre-trained discriminative encoder is then attached to a restorative decoder, forming a skip-connected encoder-decoder, for further joint discriminative and restorative learning, and finally, the pre-trained encoder-decoder is associated with an adversarial encoder for final full discriminative, restorative, and adversarial learning.

Extensive experiments discussed below demonstrate that the stepwise incremental pre-training stabilizes United models pre-training, resulting in significant performance gains and annotation cost reduction via transfer learning in nine target tasks, ranging from classification to segmentation, across diseases, organs, datasets, and modalities. This performance improvement is attributed to the synergy of the three SSL ingredients in the United framework unleashed through stepwise incremental pre-training.

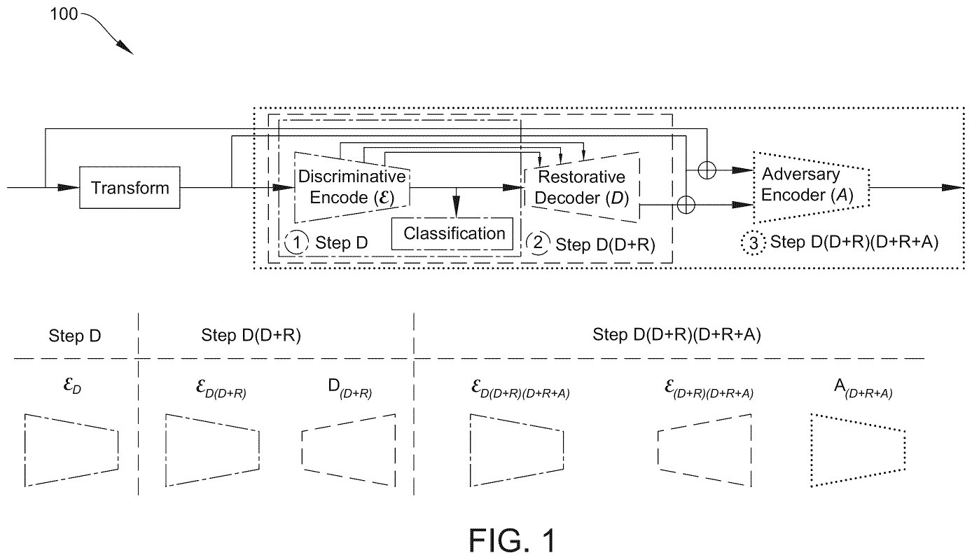

FIG. 1 depicts an exemplary “United” model 100 with stepwise incremental pretraining: D(D+R)(D+R+A). The model consists of three distinct components: a discriminative encoder E, a restorative decoder D, and an adversary encoder A, where the discriminative encoder and the restorative decoder are skip connected, forming an encoder-decoder (E, D), according to embodiments of the invention.

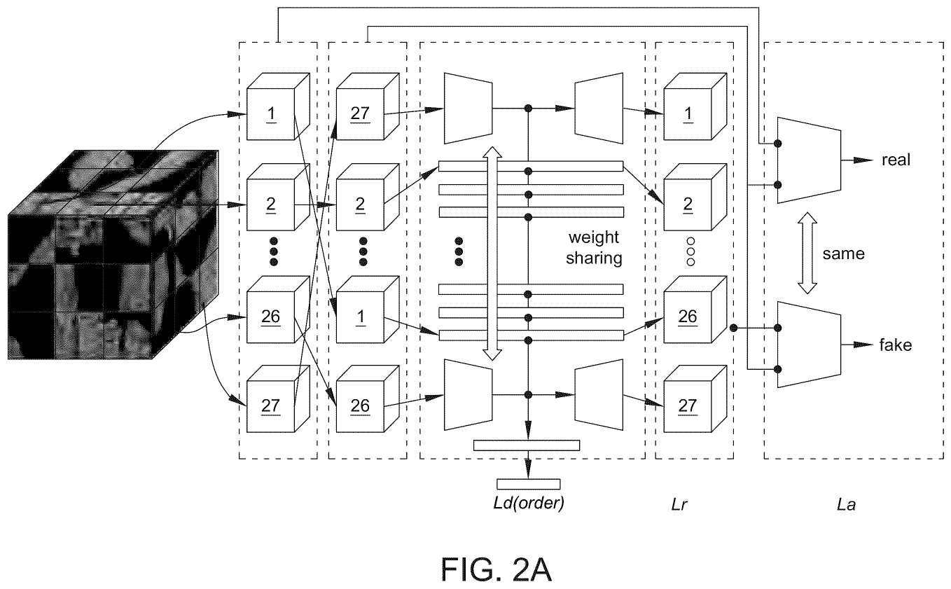

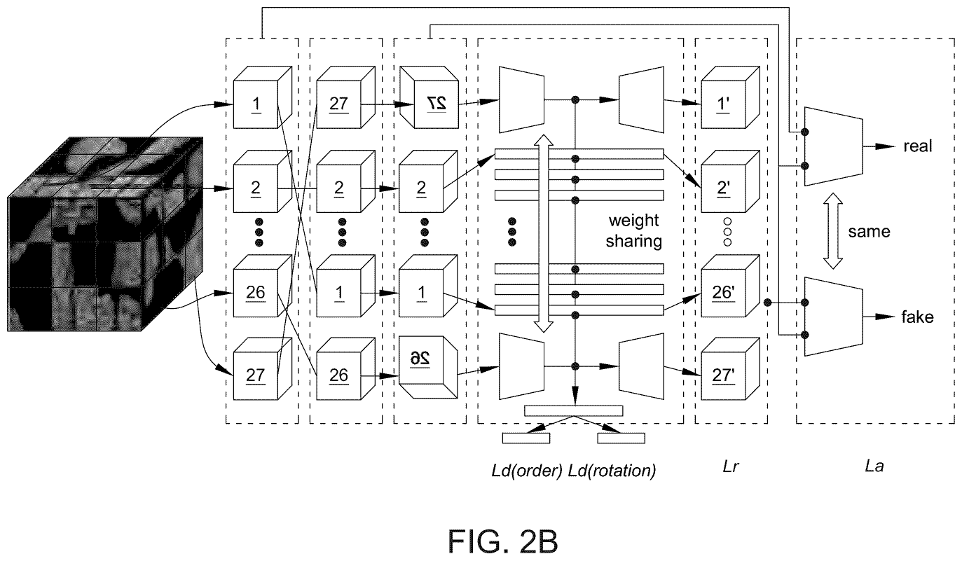

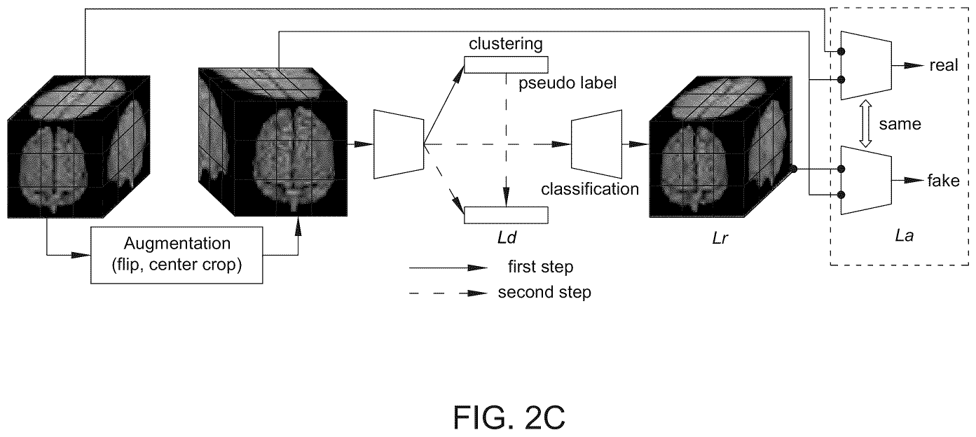

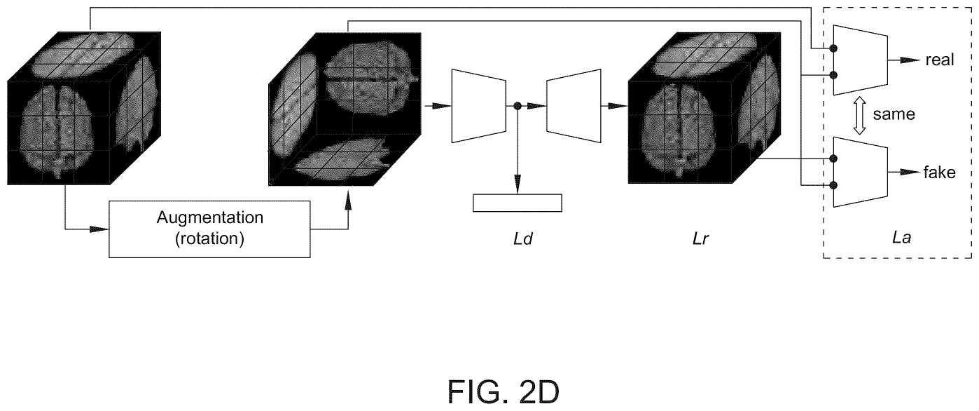

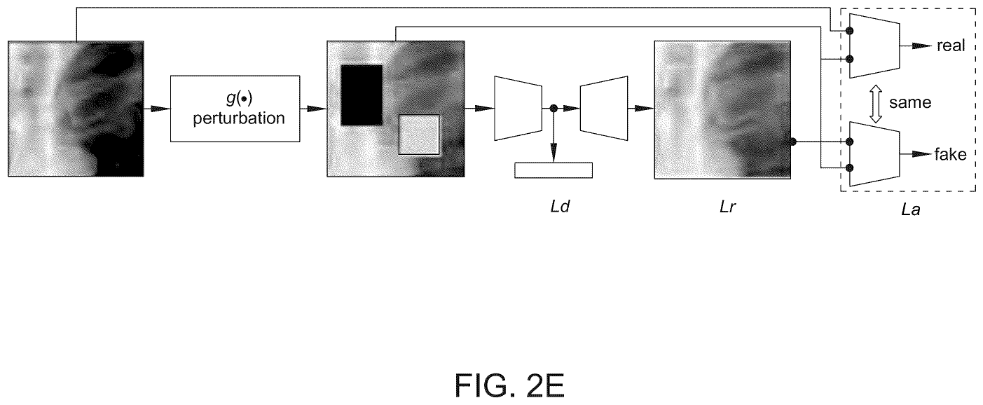

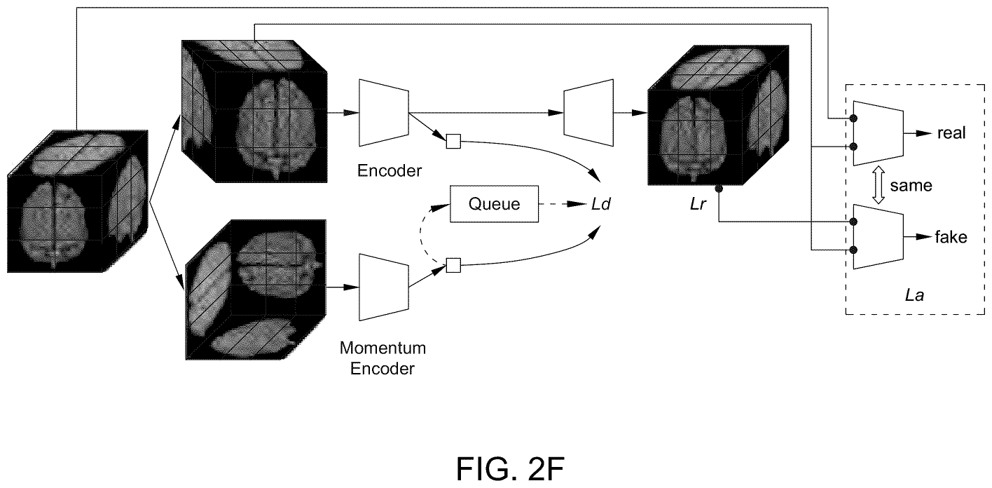

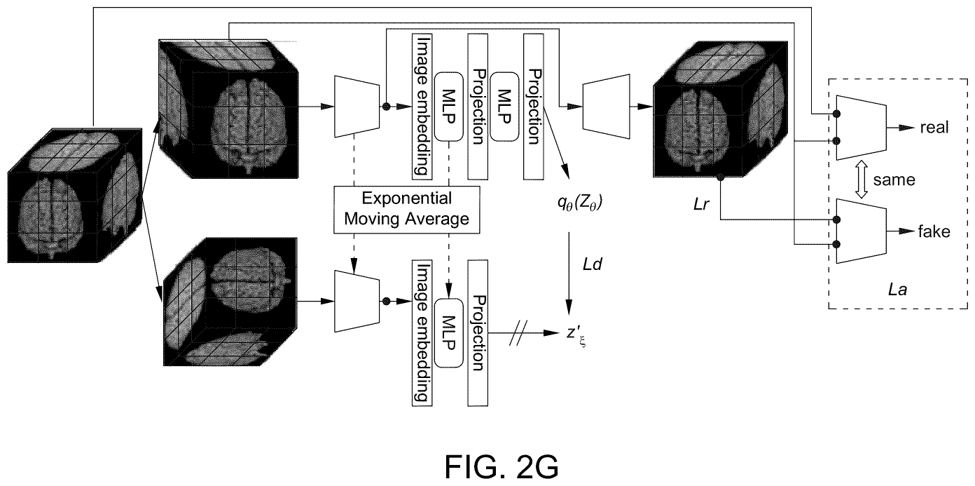

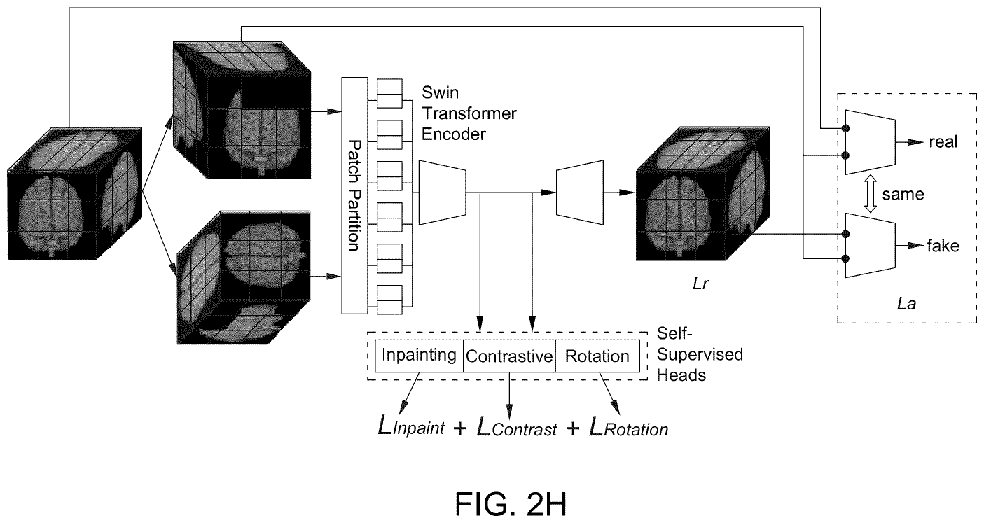

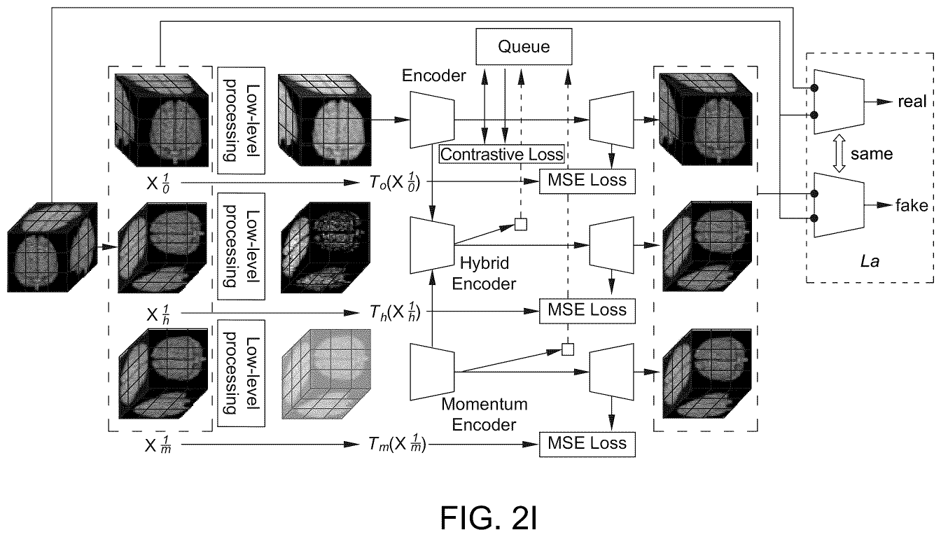

FIGS. 2A, 2B, 2C, 2D, 2E, 2F, 2G, 2H and 2I depict a nine revised SSL methods, including in FIG. 2A, Jigsaw, FIG. 2B, Rubik's Cube, FIG. 2C, Deep Clustering, FIG. 2D, Rotation, FIG. 2E, TransVW, FIG. 2F, MoCo, FIG. 2G, BYOL, FIG. 2H, Swin UNTRE, and FIG. 2I, RCRL, within the exemplary United framework, according to disclosed embodiments. The original Jigsaw, Deep Clustering, Rotation, MoCo were proposed for 2D image analysis employing discriminative learning alone and provided only pre-trained encoders; therefore, in the disclosed United framework (refer again to FIG. 1), the techniques depicted at FIGS. 2A-2I are augmented with two new components for restorative learning and adversarial learning and re-implemented in 3D. The Rubik's Cube technique is re-implemented and augmented with a new learning ingredient as shown at FIG. 2B. The original TransVW is supplemented with adversarial learning as shown at FIG. 2C. The nine revised methods provide all three learned components: discriminative encoders, restorative decoders, and adversary encoders, which are transferable to target classification and segmentation tasks.

To overcome the United model complexity and pre-training difficulty, a strategy, called D(D+R)(D+R+A), is used to incrementally train the three components in a stepwise fashion: (1) Step D trains a discriminative encoder E_{Ø}, where Ø indicates that encoder E is randomly initialized, via discriminative learning (i.e., D), leading to a pre-trained discriminative encoder E_{D}; (2) Step D(D+R) attaches the pre-trained discriminative encoder E_{D} to a randomly-initialized restorative decoder D_{Ø} for further joint discriminative and restorative learning (i.e., D+R), yielding a pre-trained discriminative encoder E_{D(D+R)} and a pre-trained restorative decoder D_{(D+R)}; (3) Step D(D+R)(D+R+A) and associates the pre-trained encoder-decoder (E_{D(D+R)}, D_{(D+R)}) with a randomly-initialized adversarial encoder A_{Ø} for final full discriminative, restorative, and adversarial learning (i.e., D+R+A), resulting in a pre-trained discriminative encoder E_{D(D+R)(D+R+A)}, a pre-trained restorative decoder E_{(D+R)(D+R+A)}, and a pre-trained adversarial encoder A_{(D+R+A)}. This stepwise incremental pre-training has proven to be reliable across multiple SSL methods (refer to FIGS. 2A-2I) for a variety of target tasks across diseases, organs, datasets, and modalities.

### INTRODUCTION

Self-supervised learning (SSL) pre-trains generic source models without using expert annotation, allowing the pre-trained generic source models to be quickly fine-tuned into high-performance application-specific target models to minimize annotation cost. Prior known SSL methods may employ just one of the following three learning ingredients: (1) discriminative learning, which pre-trains an encoder by distinguishing images associated with (computer-generated) pseudo labels; (2) restorative learning, which pre-trains an encoder-decoder by reconstructing original images from their distorted versions; and (3) adversarial learning, which pre-trains an additional adversary encoder to enhance restorative learning. It has already been demonstrated that combining self-supervised discriminative methods and restoration enhances network performance in both classification and segmentation tasks. Further, it has been demonstrated that reconstructive method is further enhanced by adversarial learning. It is contemplated that combining all three components-discriminative, restorative, and adversarial learning-yields the best performance. However, no framework or implementation has successfully integrated these three learning ingredients into a unified pre-trained AI model.

Attempts at integrating the three learning ingredients into one single framework for collaborative learning yield three learned components: a discriminative encoder, a restorative decoder, and an adversary encoder (refer again to FIG. 1). However, such integration inevitably increases model complexity and pre-training difficulty, raising the questions of how to optimally pre-train such complex generic models and how to effectively utilize pre-trained components for target tasks.

In answer to these two questions, nine prominent SSL methods for 3D imaging were redesigned, including Rotation, Jigsaw, Rubik's Cube, Deep Clustering, TransVW, Momentum Contrast (known as “MoCo”), BYOL (“Bootstrap Your Own Latent”), Swin UNETR (Swin UNEt TRansformers) and PCRL (“Preservational Contrastive Representation Learning”). Among these methods, Rotation, Jigsaw, and Rubik's Cube are classic discriminative methods. Deep Clustering is a classic clustering method. TransVW and PCRL are methods that integrate both discriminative and restorative approaches. MoCo and BYOL are contrastive methods. Swin UNETR is a transformer-based model that incorporates contrastive, restorative, and discriminative methods. With these methods, the aim is to encompass all components and models of SSL, emphasizing the generality of the novel approach described herein. These nine methods were then each formulated into a single custom configured framework which is described herein and called the “United” model or the “United” framework (refer again to FIGS. 2A-2I), as the model successfully unites the discriminative, restorative, and adversarial learning methodologies, according to embodiments of the invention.

Pre-training United models, with all three components together, directly from scratch is unstable; therefore, various training strategies were investigated and a stable solution was discovered. Specifically, stepwise incremental pre-training was identified as a viable solution to the stability problem.

An example of such pre-training is as follows: first training a discriminative encoder via discriminative learning, called Step D, then attaching the pre-trained discriminative encoder to a restorative decoder (i.e., forming an encoder-decoder) for further combined discriminative and restorative learning, called Step D(D+R), and finally associating the pre-trained auto-encoder with an adversarial-encoder for the final full discriminative, restorative, and adversarial training, called Step D(D+R)(D+R+A). This stepwise pre-training strategy provides the most reliable performance across most target tasks evaluated in this work encompassing both classification and segmentation (refer to the discussion below in the context of Tables 2, 3, 4, 5, and 7A-7C).

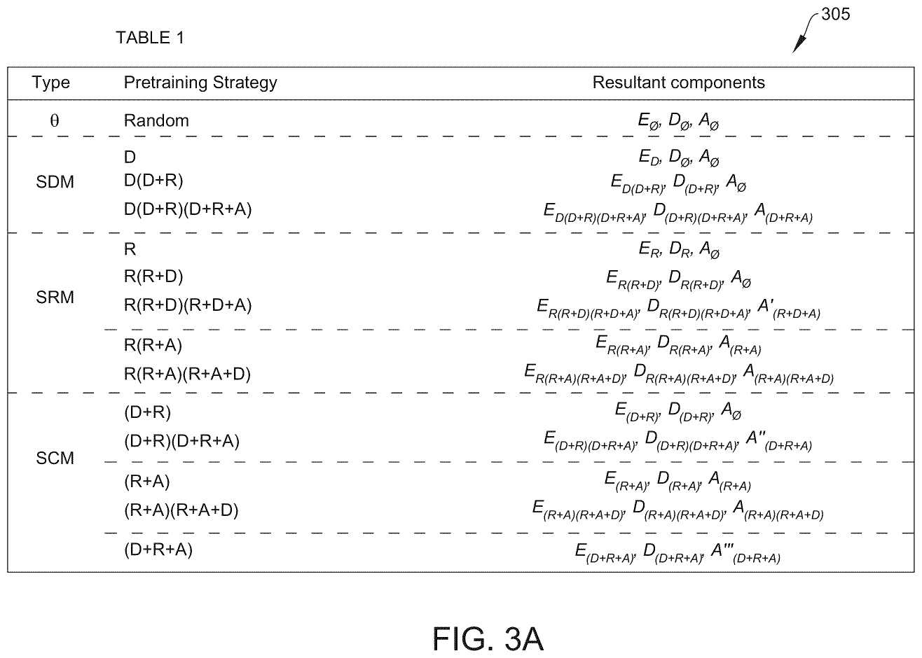

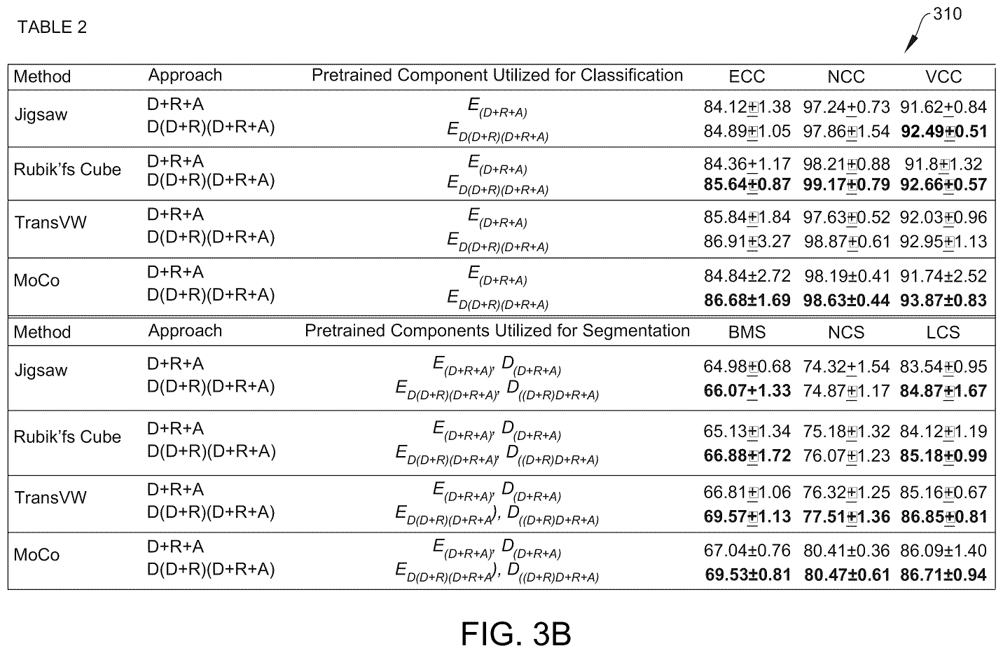

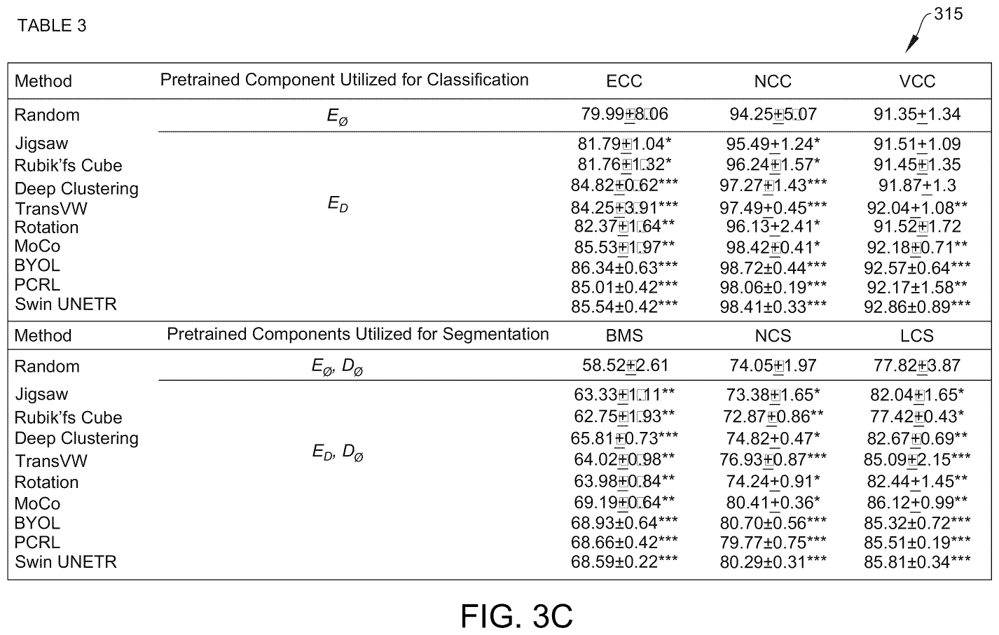

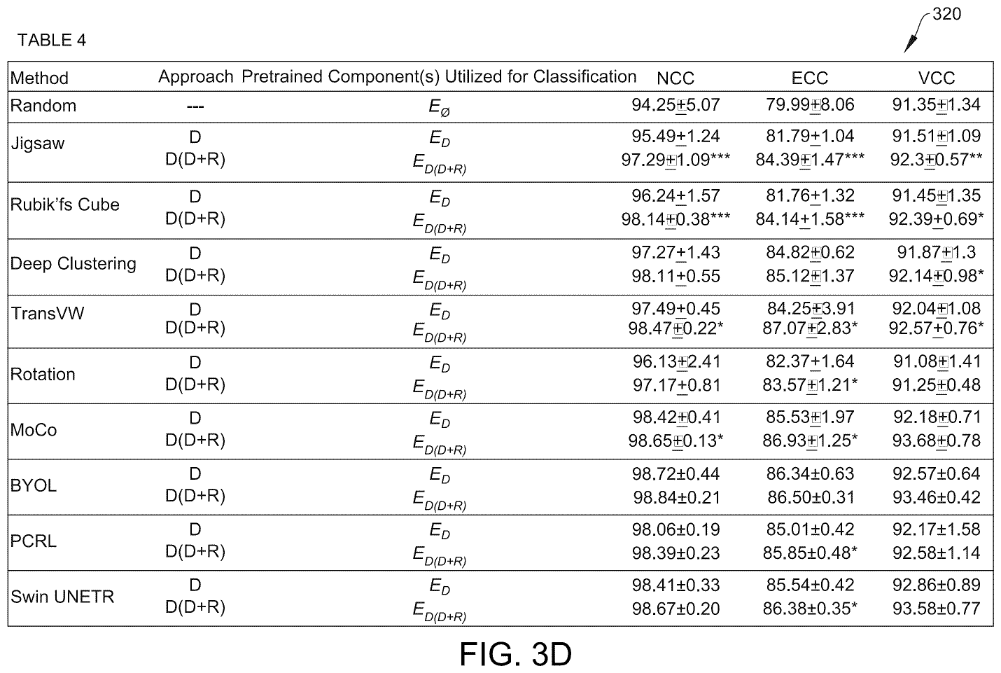

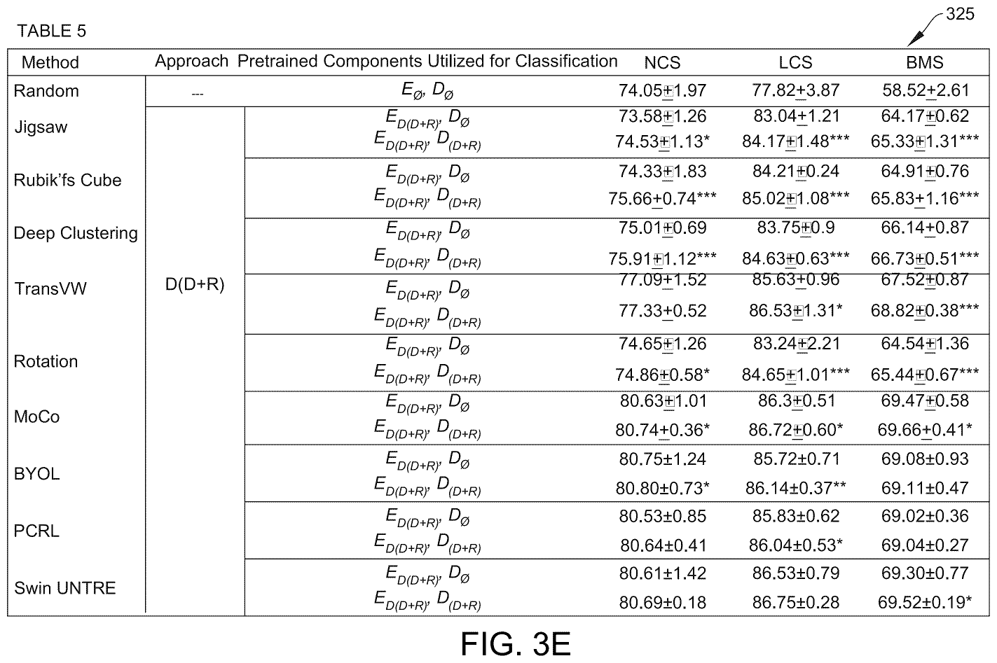

FIG. 3A presents Table 1, as set forth at element 305, depicting that, when training a United model continually component-by-component, the stepwise incremental pre-training may choose to train the components in different sequences, leading to various pre-training strategies. These strategies can be identified as three types: starting training from the discriminative methods (SDM), start training from the reconstructive methods (SRM), and start training with combined methods (SCM). Table 1 lists the pre-training strategies and their corresponding categories according to all valid component sequences and associates each strategy with its resultant components. For generality, the random initialization was considered as a pre-training strategy, which “generates” randomly-initialized discriminative encoders E_{Ø}, restorative decoders D_{Ø}, and adversary encoders A_{Ø}. For completeness, all components were listed with subscripts indicating their pre-training strategies. Those components that cannot be trained by a particular strategy are indicated explicitly with subscript Ø. These pre-training strategies are evaluated in Tables 7A, 7B, and 7C, as discussed below.

FIG. 3B presents Table 2, as set forth at element 310, showing how the strategy D(D+R)(D+R+A) always outperforms Strategy D+R+A on all nine target tasks. The mean and standard deviation are included from ten runs and an independent two-sample t-test between the two strategies. The text in boldface type font indicates when they are significantly different at p=0.05 level.

FIG. 3C presents Table 3, as set forth at element 315, showing how discriminative learning enhances discriminative encoders for classification and segmentation tasks. The mean and standard deviation (mean±s.d.) are reported across ten trials, along with the statistical analysis (*p<0.5, **p<0.1, ***p<0.05) with only discriminative pre-training for nine self-supervised learning methods. With discriminative pre-training, the performance gains were observed across target tasks with exceptions in NCS for Jigsaw and Rubik's Cube and in LCS for Rubik's Cube, where the performance of the pre-trained model is worse than random initialization due to possible incompatibilities between pre-training and targets.

FIG. 3D presents Table 4, as set forth at element 320, showing how incremental restorative pre-training combined with continual discriminative learning (i.e., Strategy D(D+R)) enhances discriminative encoders for classification tasks. The mean and standard deviation (mean±s.d.) are reported across ten trials, along with the statistic analysis (*p≤0.05, **p≤0.01, ***p≤0.001) with and without incremental restorative pre-training for nine self-supervised learning methods. With Strategy D(D+R)), the performance gains from E_{D(D+R)} were consistent for all target tasks in comparison with E_{D}.

FIG. 3E presents Table 5, as set forth at element 325, showing how incremental restorative pre-training combined with continual discriminative learning (i.e., Strategy D(D+R)) directly boost target segmentation tasks. Statistic analysis (*p<0.5, **p<0.1, ***p<0.05) was conducted between using incremental restorative pre-trained decoder (D_{(D+R)}) and using random decoder (D_{Ø}). With Strategy D(D+R), the segmentation performance gains from (E_{D(D+R)}, D_{(D+R)}) were consistent for all target tasks in comparison with (E_{Ø}, D_{Ø}) and (E_{D(D+R)}, D_{Ø}). The evaluation results use (E_{D(D+R)}, D_{Ø}) to indicate that pre-trained E_{D(D+R)} is attached with a randomly-initialized decoder D_{Ø} to form a U-Net for segmentation without using pre-trained D_{(D+R)}, despite being known, to highlight the capability of D_{(D+R)} in boosting target segmentation performance.

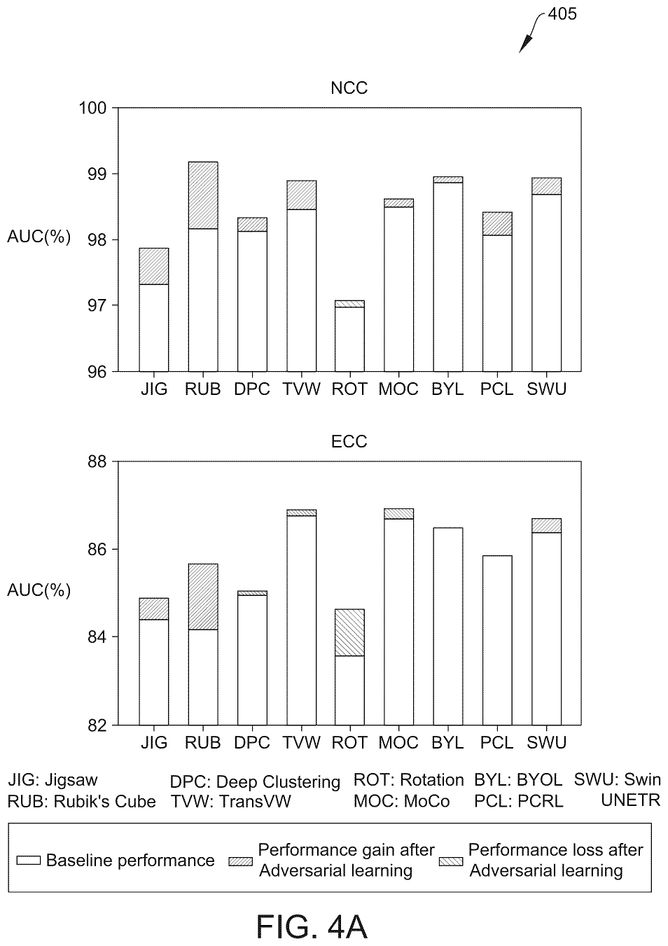

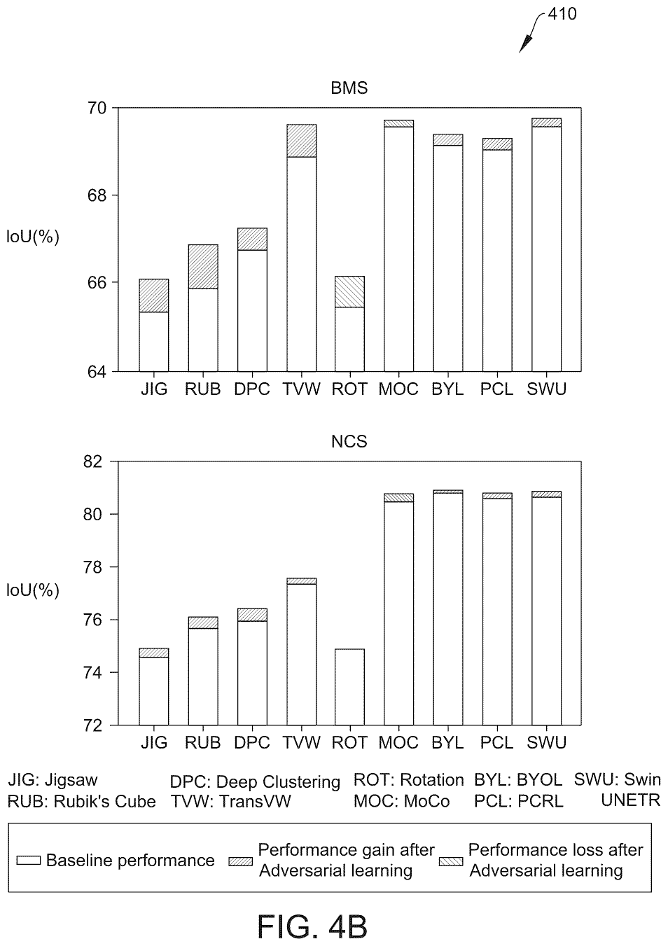

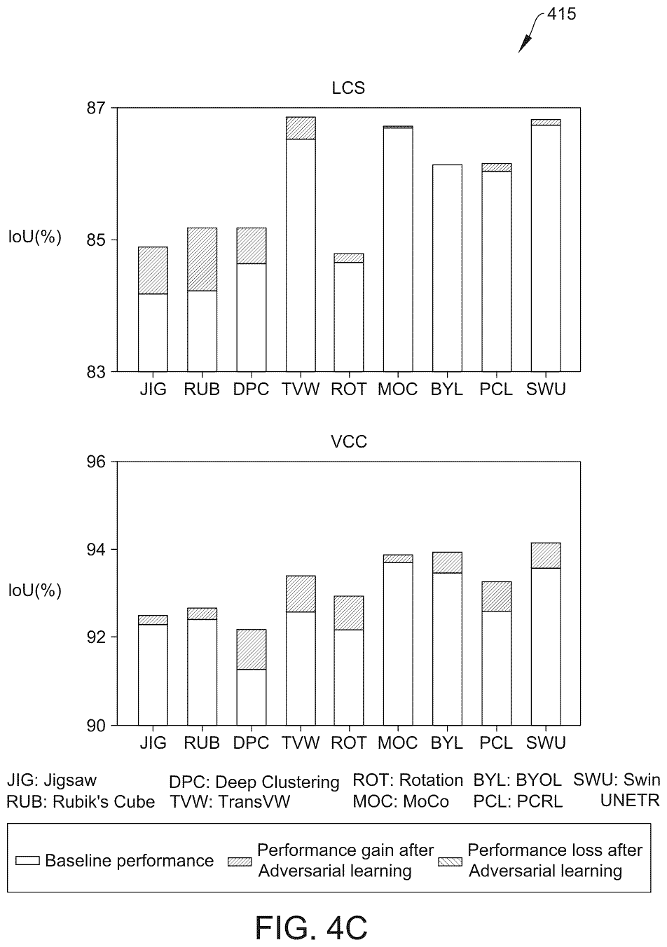

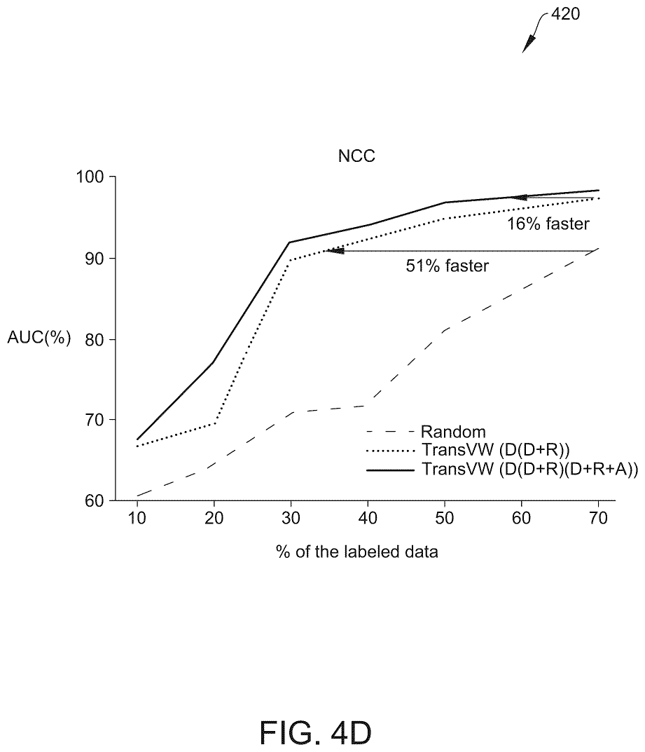

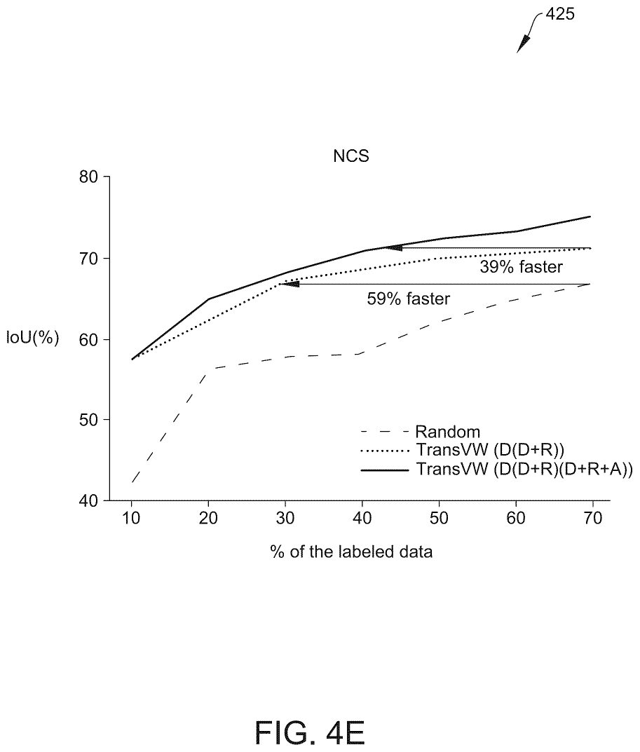

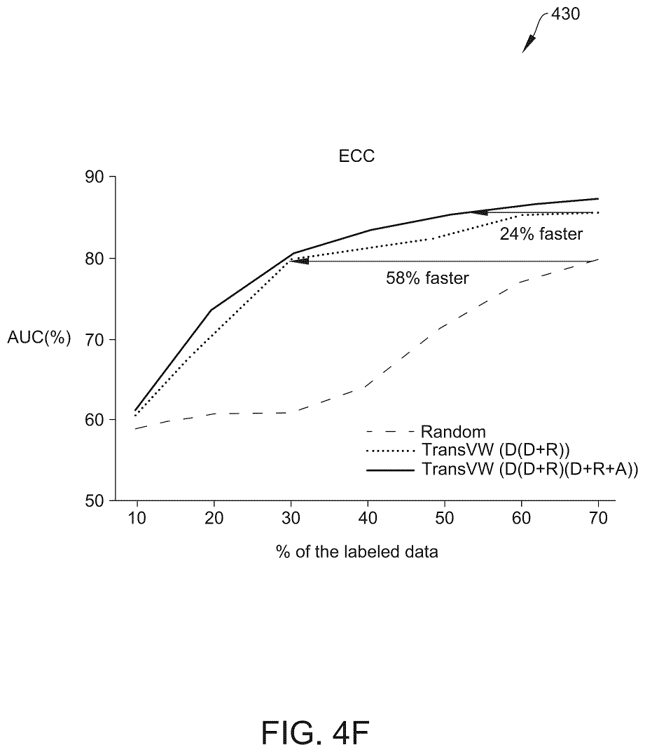

Through extensive experiments, it was observed that (1) discriminative learning alone (i.e., Step D) significantly enhances discriminative encoders on target classification tasks (e.g., +3% and +4% AUC improvement for lung nodule and pulmonary embolism false positive reduction as shown in Table 3) relative to training from scratch; (2) in comparison with (sole) discriminative learning, incremental restorative pre-training combined with continual discriminative learning (i.e., Step D(D+R)) enhances discriminative encoders further for target classification tasks (e.g., +2% and +4% AUC improvement for lung nodule and pulmonary embolism false positive reduction as shown in Table 3) and boosts encoder-decoder models for target segmentation tasks (e.g., +3%, +7%, and +5% IoU improvement for lung nodule, liver, and brain tumor segmentation as shown in Table 5); and (3) compared with Step D(D+R), the final stepwise incremental pre-training (i.e., Step D(D+R)(D+R+A)) generates sharper and more realistic medical images (e.g., FID decreases from 427.6 to 251.3 as shown in Table 6) and further strengthens each component for representation learning, leading to considerable performance gains (see FIGS. 4A, 4B, and 4C) and annotation cost reduction (e.g., 28%, 43%, and 26% faster for lung nodule false positive reduction, lung nodule tumor segmentation, and pulmonary embolism false positive reduction as shown in FIGS. 4D, 4E, and 4F) for nine target tasks across diseases, organs, datasets, and modalities.

Prior attempts have been made to combine discriminative, restorative, and adversarial learning, but the novel methodology and new findings described herein according to embodiments of the invention further extend upon those attempts, and more importantly, the methods disclosed herein significantly differ from those prior attempts which were more concerned with contrastive learning (e.g., MoCo-v2, Barlow Twins, and SimSiam) and focused on 2D medical image analysis. By contrast, the methodologies described herein according to the disclosed embodiments focus on 3D medical imaging by redesigning nine popular SSL methods beyond contrastive learning.

The use of TransVW augmented with an adversarial encoder are based on the experiments described herein. Furthermore, the following disclosure focuses on a stepwise incremental pre-training to stabilize United model training, revealing new insights into synergistic effects and contributions among the three learning ingredients.

Thus, at least the following advantages are provided by the novel methodologies which are described herein. First, a stepwise incremental pre-training strategy is provided that stabilizes United models' pre-training and unleashes the synergistic effects of the three SSL ingredients. Second, a collection of pre-trained United models is provided that integrate discriminative, restorative, and adversarial learning into a single framework for 3D medical imaging, encompassing both classification and segmentation tasks. And third, a set of extensive experiments were conducted that demonstrate how various pre-training strategies benefit target tasks across diseases, organs, datasets, and modalities.

### United Framework and Stepwise Incremental Pre-Training:

Nine prominent SSL methods were modified and specially redesigned to support the techniques set forth herein, including Rotation, Jigsaw, Rubik's Cube, Deep Clustering, TransVW, MoCo, BYOL, Swin UNTRE and PCRL; and each was augmented with the missing components under the disclosed United framework (refer again to FIGS. 2A-2I).

A United model (refer again to FIG. 1) is a skip-connected encoder-decoder associated with an adversary encoder. With this redesign, for the first time, all nine methods have all three SSL components for 3D medical image analysis.

Jigsaw: Jigsaw self-supervised learning is a popular technique for training deep neural networks without the need for labeled data. The 3D Jigsaw approach described herein extends upon the original idea proposed for 2D by modifying and extending it into 3D as shown in FIG. 2A. The 3D jigsaw first divides an input image into a 3×3×3 grid of 3D patches and shuffles them according to a predefined permutation. To reduce the number of classes, 1000 permutations were selected from all possible combinations using a Hamming Distance-based algorithm. Each permutation is treated as a class, and the Jigsaw puzzle is reformulated as a classification task where the model is trained to recognize the permutation ID.

Rubik's Cube: like the Jigsaw Puzzle pretext task, Rubik's Cube predicts the relative position of sub-cubes in pretext training. As shown at FIG. 2B, it can be seen as the 3D extension of the jigsaw puzzle and naturally takes advantage of volumetric medical image data. Moreover, it is a multitask system that not only predicts the relative position of the sub-cubes but also judges whether each cube has been rotated. This method is a discriminative approach as both pretext tasks are classification problems.

Deep Clustering: Deep clustering extends traditional clustering methods by applying them within neural networks. This method simultaneously learns the parameters of the neural network and the cluster assignment of the extracted feature. It can be viewed as a discriminative method as it learns the parameters through classification tasks. The method was applied to the medical domain for 3D applications by altering the Convolutional Neural Network (CNN) architecture as illustrated in FIG. 2C.

Rotation: The rotation-based self supervised learning method teaches a CNN to recognize the rotation angle of an image without the need of human supervision. This is done by defining four possible rotation angles (0, 90, 180, and 270 degrees) and asking the network to predict which angle the image has been rotated by. Extending upon this concept, a 3D implementation of the rotation-based method is shown at FIG. 2D. The disclosed implementation builds upon and extends prior attempts by specially configuring the system to add restorative and adversarial learning to fit the rotation-based method into the United framework disclosed herein.

TransVW: TransVW is an innovative framework for self-supervised learning that leverages self-discovered visual words as the supervision signal to train a CNN using an encoder-decoder architecture with skip connections and a classification head. The self-discovered visual words are used as the supervision signal. Then, through self-classification, the model is trained to classify each of the visual words. TransVW is like deep clustering, but rather than using the entire image to form clusters, the self-discovering process only considers the patches extracted from the same coordinate across the similar images, as shown at FIG. 2E.

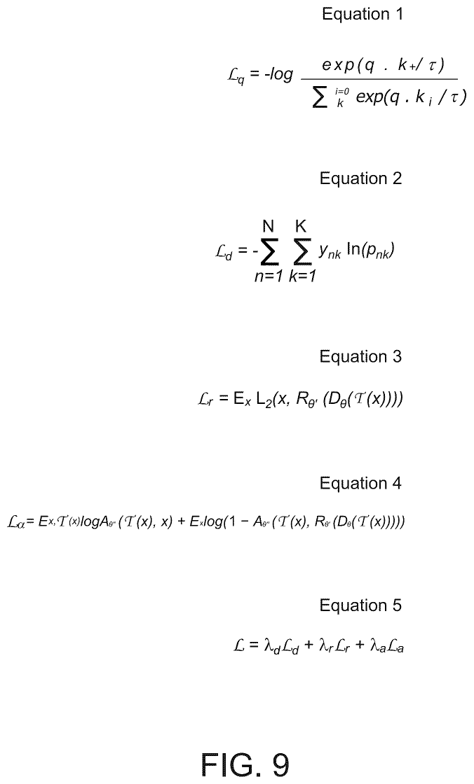

MoCo: The MoCo (Momentum Contrast) technique is an unsupervised visual representation learning technique that makes use of contrastive loss, as shown at FIG. 2F. It includes two encoders, the standard encoder and the momentum encoder. The momentum encoder computes mini-batches and stores them in a queue. The encoders then take the same image with different augmentations and compute the similarity between this encoding and the ones in the queue. The standard encoder is updated using back-propagation, while the momentum encoder is updated through a linear interpolation of the earlier standard encoders. The training object is formulated using the InfoNCE loss function, which maximizes the similarity between the positive pair and minimizes the similarity between the negative pairs, according to Equation 1, as follows and as provided along with subsequent equations discussed below in FIG. 9:

$${ℒ}_{q} = - log \frac{exp ( q \cdot {k}_{+} / τ )}{{\sum }_{K}^{i = 0} exp ( q \cdot {k}_{i} / τ )} .$$

BYOL: BYOL utilizes a pair of neural networks known as the online and target networks, which collaborate and mutually enhance their learning processes. The online network is trained to predict the target network representation of an image from an augmented view, with the input image presented under a different augmentation. Simultaneously, the target network undergoes updates based on a gradual average of the online network. Notably, BYOL diverges from conventional training methods by not requiring negative samples and abstaining from contrastive loss during its training process. For a given input image x, BYOL generates two augmented views v≙t(x) and v′≙t′(x). From the initial augmented view v, the online network produces a representation y_{θ}≙f_{θ}(v) and a corresponding projection z_{θ}≙g_{θ}(y). Simultaneously, the target network generates y′_{ξ}≙f_{ξ}(v′) and the associated target projection z′ξ≙g_{ξ}(y′). The loss is computed using the mean squared error between these two projections:

$${ℒ}_{0 q} , ξ {=}^{△} {‖ {\overline{q}}_{θ} ( {z}_{θ} ) - {\overline{z}}_{ξ}^{′} ‖}_{2}^{2} = 2 - 2 \cdot \frac{〈 {q}_{θ} ( {z}_{θ} ) , {z}_{ξ}^{′} 〉}{{‖ {q}_{θ} ( {z}_{θ} ) ‖}_{2} \cdot {‖ {z}_{ξ}^{′} ‖}_{2}}$$

PCRL: PCRL combines contrastive and generative self-supervised methods to address the challenge of preserving comprehensive contextual cues in medical images. An innovative aspect involves a generative pretext task that recovers a transformed input using a designated indicator vector, promoting the encoding of richer information. Additionally, a mix-up strategy is employed to diversify image restoration.

Swin UNETR: Swin UNETR employs a Swin Transformer encoder for processing 3D input patches in pretext tasks. The transformer is pre-trained using self-supervised tasks like image inpainting, 3D rotation prediction, and contrastive learning, utilizing randomly cropped sub-volumes with stochastic data augmentations. The Swin Transformer extracts features at four resolutions via shifted windows for self-attention, connecting to a CNN-based decoder with skip connections at each resolution. This approach efficiently captures global and local information across layers, ensuring scalability for large-scale training.

Stepwise Incremental Pre-training: The United models were incrementally trained component-by-component in a stepwise manner, yielding three learned transferable components: discriminative encoders, restorative decoders, and adversarial encoders. The pre-trained discriminative encoder can be fine-tuned for target classification tasks; the pre-trained discriminative encoder and restorative decoder, forming a skip-connected encoder-decoder network (i.e., a U-Net), can be fine-tuned for target segmentation tasks.

Discriminative learning: Discriminative learning is a technique for training a discriminative encoder D_{θ}, where θ represents the model parameters, to predict target label y∈Y from input x∈X by minimizing a loss function for ∀x∈X defined according to Equation 2, as follows:

$${ℒ}_{d} = - {\sum }_{n = 1}^{N} {\sum }_{k = 1}^{K} {y}_{nk} ln ( {p}_{nk} ) ,$$

where N is the number of samples, K is the number of classes, and pₙₖ is the probability predicted by D_{θ} for xₙ belonging to Class k; that is, pₙ=D_{θ} (xₙ) is the probability distribution predicted by D_{θ} for xₙ over all classes. In SSL, the labels are automatically obtained based on the properties of the input data, involving no manual annotation. All nine SSL methods utilized herein have a discriminative component formulated as a classification task, while other discriminative losses can be used, such as contrastive losses in MoCo-v2, Barlow Twins, and SimSiam.

Restorative learning: Restorative learning is a technique for training an encoder-decoder (D_{θ}, R_{θ′}) to reconstruct an original image x from its distorted version T(x), where Tis a distortion function, by minimizing pixel-level reconstruction error, according to Equation 3, as follows:

$${ℒ}_{r} = {𝔼}_{x} {L}_{2} ( x , {R}_{{θ}^{′}} ( {D}_{θ} ( 𝒯 ( x ) ) ) ) ,$$

where L₂(u, v) is the sum of squared pixel-by-pixel differences between u and v.

Adversarial learning: Adversarial learning is applied for the training of an additional adversary encoder, A_{θ″}, to help the encoder-decoder (D_{θ}, R_{θ′}) reconstruct more realistic medical images and, in turn, strengthen representation learning. The adversary encoder learns to distinguish fake image pair (R_{θ′}(D_{θ}(T(x))), T(x)) from real pair (x, T(x)) via an adversarial loss, according to Equation 4, as follows:

$${ℒ}_{a} = {E}_{x , 𝒯 ( x )} log {A}_{{θ}^{′}} , ( 𝒯 ( x ) , x ) + {E}_{x} log ( 1 - {A}_{{θ}^{′}} , ( 𝒯 ( x ) , {R}_{{θ}^{′}} ( {D}_{θ} ( 𝒯 ( x ) ) ) ) ) .$$

The final objective is to combine all losses, according to Equation 5, as follows:

$$ℒ = {λ}_{d} {ℒ}_{d} + {λ}_{r} {ℒ}_{r} + {λ}_{a} {ℒ}_{a} ,$$

where λ_{d}, λᵣ, and λₐ controls the importance of each learning ingredient. A grid-search hyper-parameter optimization was performed which estimated the optimal values of λ_{d}=1, λᵣ=1, and λₐ=10.

Stepwise incremental pre-training: This technique trains a United model continually component-by-component because the model's complexity makes it difficult to train the whole model in an end-to-end fashion (i.e., all three components together directly from scratch), a strategy called D+R+A. The validation performance of this strategy fluctuates significantly during the training process. The strategy D+R+A is always outperformed by, for example, Strategy D(D+R)(D+R+A), which is illustrated in FIG. 1 and which provides the most reliable performance across most target tasks evaluated in this work (refer again to Table 2). When training a United model continually component-by-component, the stepwise incremental pre-training may follow different component sequences, leading to various pre-training strategies as summarized in Table 1. These pre-training strategies are compared in the discussion below with reference to Tables 7A, 7B, and 7C.

### Experimental Setup:

Model: The U-Net model with skip connections was utilized for the study and evaluations. This model has demonstrated state-of-the-art performance in medical imaging segmentation tasks, and its encoder part was used for classification tasks. For each of the nine methods, the model was specially redesigned to incorporate all three learning components: discriminative, restorative, and adversarial.

Fine-tuning: All experiments fine-tuned the pre-trained model end-to-end on the target transfer dataset. The datasets used for pre-training and fine-tuning are introduced below.

Datasets and Metrics: The experiments used 623 CT scans from the LUNA16 dataset to pre-train all nine of the models. The experiments used extracted sub-volumes with a size of 64×64×64 pixels. To assess the usefulness of pre-training the nine models, each was tested on nine 3D medical imaging tasks including BraTS, LUNA16, LIDC-IDRI, PE-CAD, PE-CAD (VOIR), and LiTS. These tasks are BMS (brain tumor segmentation), NCC (reducing lung nodule false positives), NCS (lung nodule segmentation), ECC (reducing pulmonary embolism false positives), VCC (reducing pulmonary embolism false positives with vessel-oriented image representation), and LCS (liver segmentation). The efficacy of the pre-trained models was calculated on the nine target tasks and reported as the AUC (Area Under the ROC Curve) for classification tasks and as the IoU (Intersection over Union) for segmentation tasks. All target tasks were executed at least 10 times, and statistical analysis was performed using the independent two-sample t-test.

FIGS. 4A, 4B, and 4C show how the incremental adversarial training in Strategy D(D+)(D+R+A) strengthens learned representation. Target task performance is generally increased following the adversarial training. Although some target tasks show a decrease, these reductions are not statistically significant, according to the t-test.

FIGS. 4D, 4E, and 4F show how the stepwise incremental pre-training D(D+R)(D+R+A) helps reduce annotation costs. As an example, for target tasks of NCC, NCS, and ECC, incremental pre-trained TransVW with Strategy D(D+R)(D+R+A) reduces the annotation cost by 28%, 43%, and 26%, respectively, in comparison with Strategy D(D+R), and by 57%, 61%, and 66%, respectively, comparison with training from scratch.

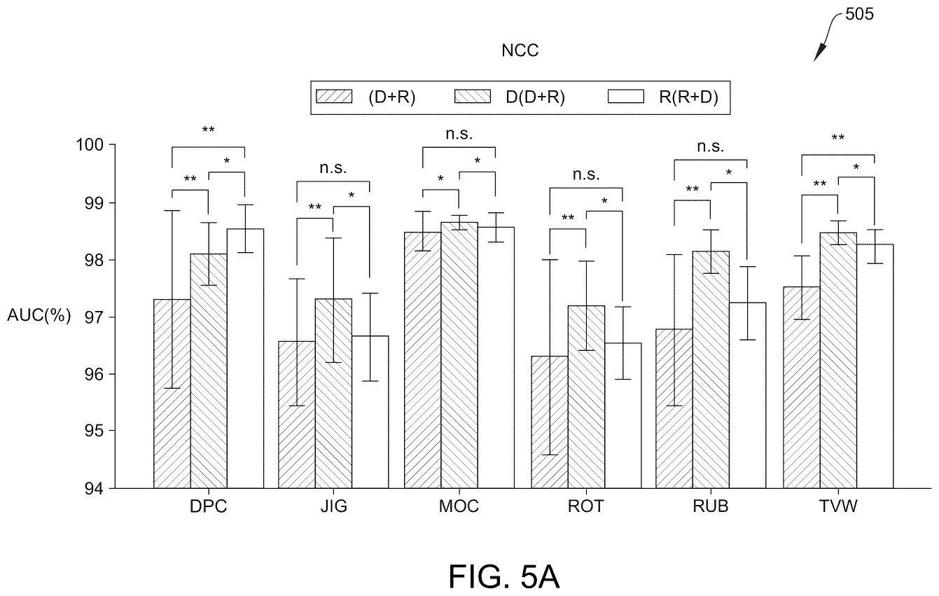

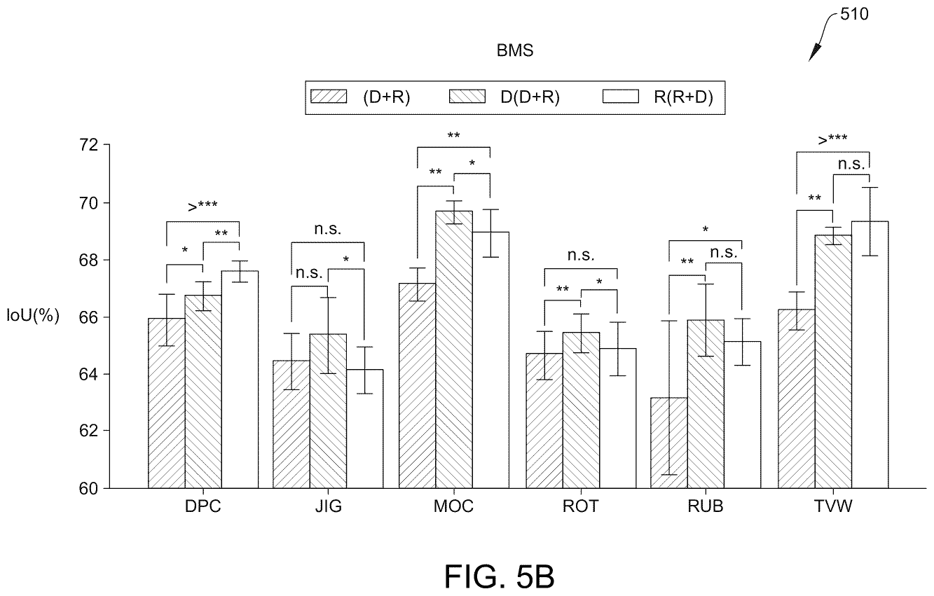

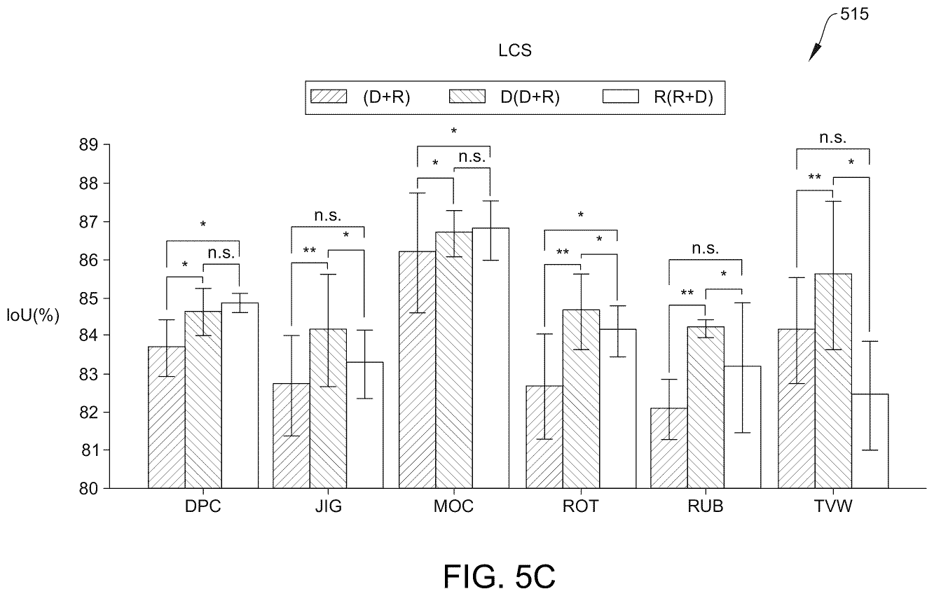

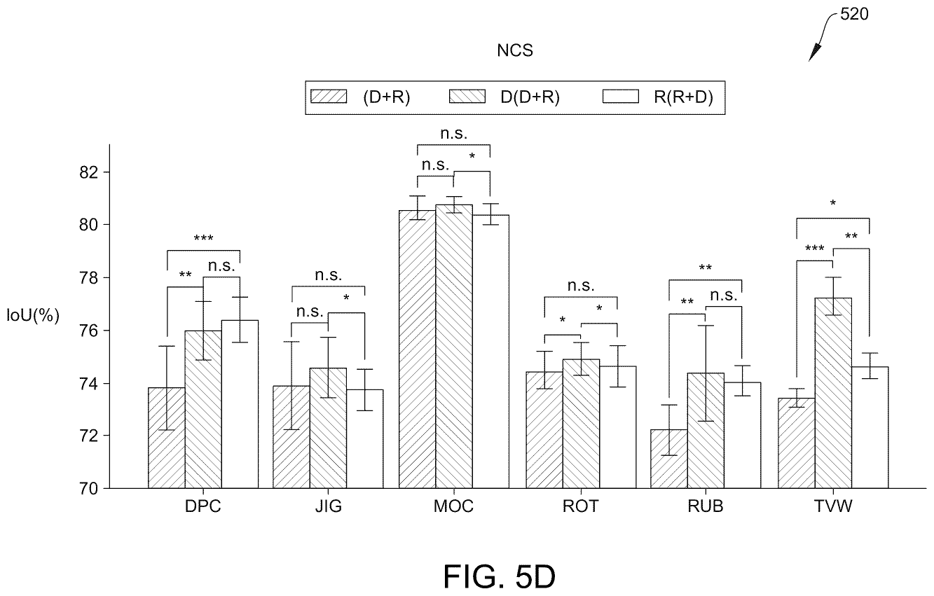

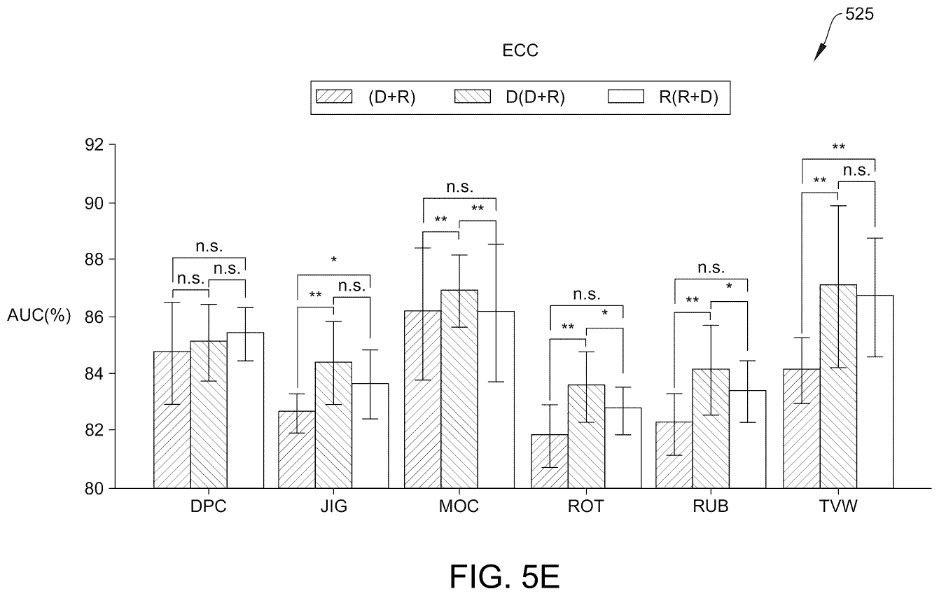

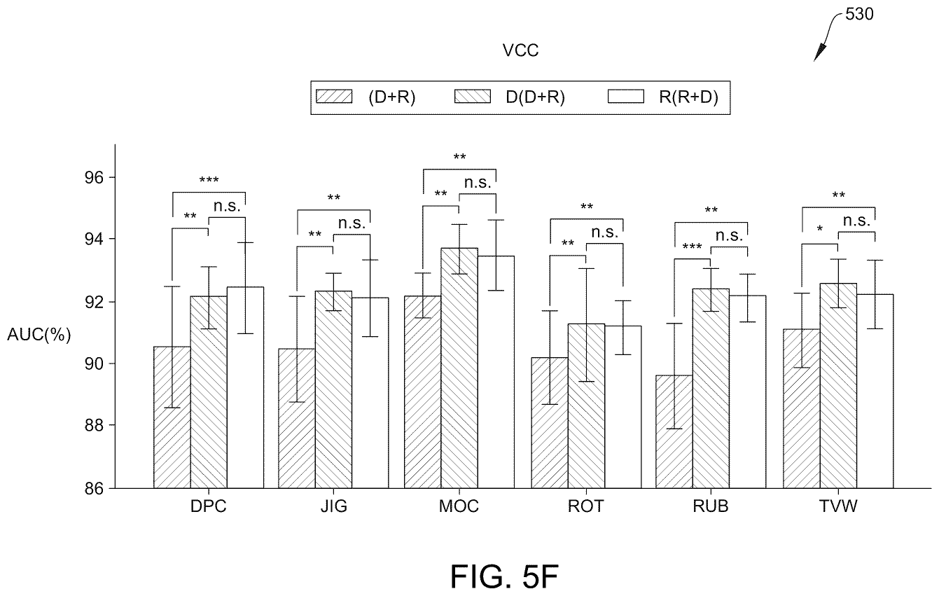

FIGS. 5A, 5B, 5C, 5D, 5E, and 5F show the different variants tested for the stepwise incremental pre-training scheme R(R+D) and D(D+R) with components D and R. Compared to the end-to-end training scheme (D+R), R(R+D) increased the target task performance most of the time. The only two performance decreases are for Jigsaw on BMS and NCS but they were not significant according to the t-test. D(D+R) always improved performance on all five target tasks compared to (D+R). For five out of nine methods, D(D+R) also had better performance on all five target tasks compared to R(R+D). The only exception is for the Deep Clustering where R(R+D) always performs better than D(D+R). It is believed that this is because for Deep Clustering, training the reconstruction (R) first helps to initialize the clusters, yielding better overall performance.

Brain tumor segmentation (BMS): The dataset, which comes from the BraTS 2018 challenge, includes 285 patients (210 HGG and 75 LGG), each with four rigorously aligned 3D MRI modalities (T1, T1c, T2, and Flair). In the 3-fold cross validation method, 95 patients comprised the test fold while 190 patients comprised the training fold. Three tumor sub-regions were annotated. Specifically, the necrotic and non-enhancing tumor core (label 1), the GD-enhancing tumor (label 4), and the peritumoral edema (label 2). Here, the background was annotated (label 0). Finally, Intersection over Union (IoU) was used to assess segmentation performance. Those with label 0 were treated as negatives and all other data as positives.

Lung nodule false positive reduction (NCC): The dataset is from LUNA16 which consists of 888 CT scans with a slice thickness less than 2.5 mm. With 445, 265, and 178 instances each, the dataset is subdivided into training, validation, and testing sets. The initial data were made available for segmenting lung nodules, but additional annotation was made available for the task of reducing false-positive results. The performance was evaluated using the Area Under the Curve (AUC) score for classifying true positives and false positive results.

Lung nodule segmentation (NCS): The dataset is made available by the Lung Image Database Consortium image collection (LIDC-IDRI) with 1088 cases consisting of lung CT scans with masked nodule locations. The training set contains 510 cases, the validation set includes 100 cases, and the testing set includes 480 cases. To train using this dataset, the CT scans were re-sampled to 1-1-1 spacing, and cubes were extracted with a size of 64×64×32. The Intersection over Union (IoU) was adopted to evaluate performance.

Pulmonary embolism false positive reduction (ECC): Evaluations made use of a database that contains 326 emboli from 121 computed tomography pulmonary angiography (CTPA) images. The evaluations used the proprietary algorithm-based PE candidate generator, which yielded a total of 687 true positives and 5,568 false positives. The dataset was then split into a training dataset and a testing dataset. The training dataset contains 434 true positive PE candidates and 3,406 false positive PE candidates. The testing dataset contains 253 true positive PE candidates and 2,162 false positive PE candidates, both at the patient-level. The candidate level AUC was calculated for distinguishing true and false positive results to facilitate an accurate comparison with the previous study.

Pulmonary embolism false positive reduction with vessel-oriented image representation (VCC): In this task, the evaluations focus on using vessel-oriented image representation (VOIR) to improve the accuracy of image representations of PE candidates. By aligning the image planes with the vessel longitudinal axis, the VOIR approach maximizes the visualization of pulmonary arterial filling defects and generates more accurate representations of PE candidates. The evaluations further extend the VOIR into 3D and evaluate the performance of all nine methods on the false positive reduction task by calculating the candidate level AUC.

Liver segmentation (LCS): A total of 130 labeled CT scans from the MICCAI, LiTS Challenge dataset were divided into subgroups for training (100 patients), validation (15 patients), and testing (15 patients). Two distinct labels, liver and lesion, were provided by the ground truth segmentation. The evaluations used Intersection over Union (IoU) to assess segmentation performance in the studies, only regarding the liver as a “positive class,” and all other classes as “negative class”.

### Experiments and Results:

In this section, the importance of the incremental pre-training strategy in the United framework is investigated, supported by discussion regarding how to utilize each component in the United framework for downstream tasks.

Discriminative Encoders (ED) are useful for both classification and segmentation tasks: The discriminative encoders were trained using nine SSL methods and they were applied to nine target tasks. The discriminative learning significantly enhances encoders in both classification and segmentation tasks, as shown in Table 3. Specifically, compared with the random initialization, the Deep Clustering method improved NCC, ECC, NCS, LCS, BMS, and VCC by AUC scores of 3.0%, 4.8%, 0.8%, 4.8%, 7.3%, and 0.5%, respectively. Similarly, TransVW improves the target tasks by 3.2%, 4.3%, 2.9%, 7.2%, 5.5%, and 0.7%, Rotation by 1.9%, 2.4%, 0.2%, 4.6%, 5.5%, and 0.2%, MoCo by 4.2%, 5.5%, 6.4%, 8.3%, 10.6%, and 0.8%, BYOL by 0.1%, 0.2%, 0.1%, 0.8%, 0.2%, and 0.9%, PSL by 0.3%, 0.8%, 0.9%, 0.5%, 0.4%, and 0.4%, and SWU by 0.3%, 0.87%, 0.4%, 0.9%, 0.9%, 0.7%. The Jigsaw method improved NCC, ECC, LCS, BMS, and VCC by AUC scores of 1.3%, 1.8%, 4.2%, 3.8%, and 0.2%, respectively. The Rubik's Cube method improved in NCC, ECC, BMS, and VCC by AUC scores of 2.0%, 1.8%, 4.2%, and 0.1%, respectively.

Incremental restorative pre-training combined with continual discriminative learning (i.e., Strategy D(D+R)) further enhances discriminative encoders for classification tasks: After pre-training discriminative encoders, restorative decoders were appended to the end of the encoders and continue to pre-train them together. The incremental restorative learning significantly enhances encoders in classification tasks, as shown in Table 4. Specifically, compared with the prior known methods, the incremental restorative learning improves Jigsaw by AUC scores of 1.9%, 2.6%, and 0.8% in NCC, ECC, and VCC; similarly, it improves Rubik's Cube by 1.9%, 2.4%, and 0.9%, Deep Clustering by 0.9%, 0.3%, and 0.3%, Trans VW by 1.0%, 2.9%, and 0.5%, Rotation by 1.0%, 1.2%, and 0.2%, MoCo by 0.2%, 1.4%, and 1.5%, BYOL by 0.1%, 0.2%, and 0.9%, PCRL by 0.3%, 0.8%, and 0.4%, and Swin UNTRE by 0.3%, 0.8%, and 0.7%. The discriminative encoders were enhanced because they learn global features along with fine-grained features through incremental restorative learning.

Incremental restorative pre-training combined with continual discriminative learning (i.e., Strategy D(D+R)) directly boosts target segmentation tasks: Most state-of-the-art segmentation methods do not pre-train their decoders, but instead initialize them at random. Table 5 shows that the random decoders are suboptimal, while incremental pre-trained restorative decoders can significantly improve target segmentation tasks. Specifically, compared with the D methods, the incremental pre-trained restorative decoder improves Jigsaw by 1.2%, 2.1% and 2.0% IoU improvement in NCS, LCS and BMS, respectively. Similarly, it improves Rubik's Cube by 2.8%, 7.6%, and 3.1%; Deep Clustering by 1.1%, 2.0%, and 0.9%; Trans VW by 0.4%, 1.4%, and 4.8%; Rotation by 0.6%, 2.2% and 1.5%; MoCo by 0.1%, 0.4%, and 0.2%, BYOL by 0.2%, 0.4%, and 0.1%, PCRL by 0.1%, 0.2%, and 0.1%, and Swin UNTRE by 0.1%, 0.2%, and 0.2%. The consistent performance gains indicate that a wide variety of target segmentation tasks benefit from the incremental pre-trained restorative decoders.

Strategy D(D+R)(D+R+A) strengthens representation learning and reduces annotation costs: Quantitative measurements shown in Table 6 reveal that adversarial training can generate sharper and more realistic images in the restoration proxy task. More importantly, it was found that adversarial training also makes a significant contribution to pre-training. First, as shown in FIG. 3, adding adversarial training can benefit most target tasks, particularly segmentation tasks. The incremental adversarial pre-training improves Jigsaw by AUC scores of 0.3%, 0.7%, and 0.7% in NCS, LCS, and BMS, respectively. Similarly, it improves Rubik's Cube by 0.4%, 1.0%, and 1.0%; Deep Clustering by 0.5%, 0.5%, and 0.5%; TransVW by 0.2%, 0.3%, and 0.8%; Rotation by 0.1%, 0.1%, and 0.7%, BYOL by 0.1%, 0.1%, and 0.1%, PCRL by 0.1%, 0.1%, and 0.1%, and Swin UNTRE 0.3%, 0.2%, and 0.2%. Additionally, incremental adversarial pre-training improves performance on small data regimes. FIGS. 4D, 4E, and 4F show that incremental adversarial pre-trained TransVW can reduce the annotation cost by 28%, 43%, and 26% on NCC, NCS, and ECC, respectively, compared with Trans VW.

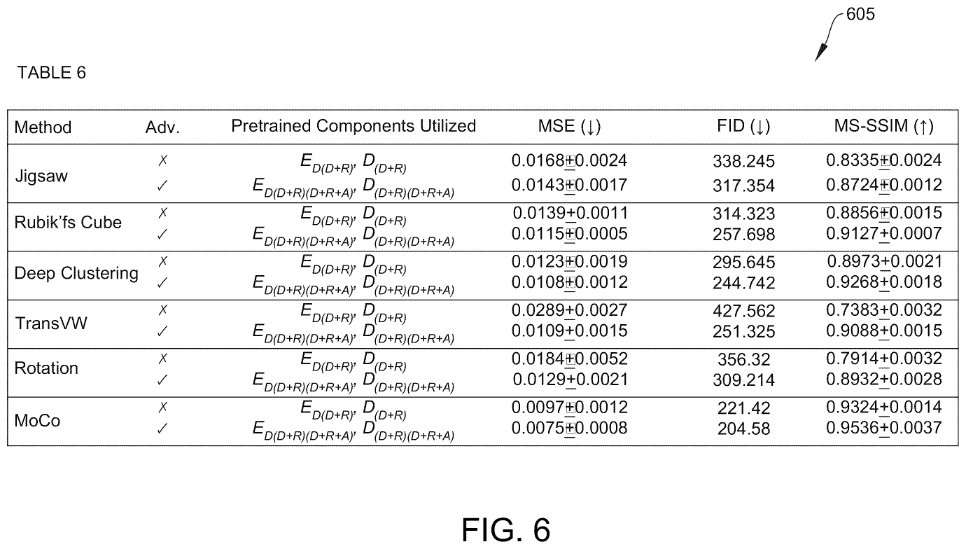

FIG. 6 depicts Table 6, as set forth at element 605, and shows how the final stepwise incremental pre-training (Step D(D+R)(D+R+A)) generates sharper and more realistic images for restoration tasks. After further adversarial training, the MSE and FID scores for each of the nine approaches all declined, suggesting that the produced images' distribution had moved closer to the original one. The MS-SSIM score increased after the adversarial training, indicating the generated images were structurally like the original one.

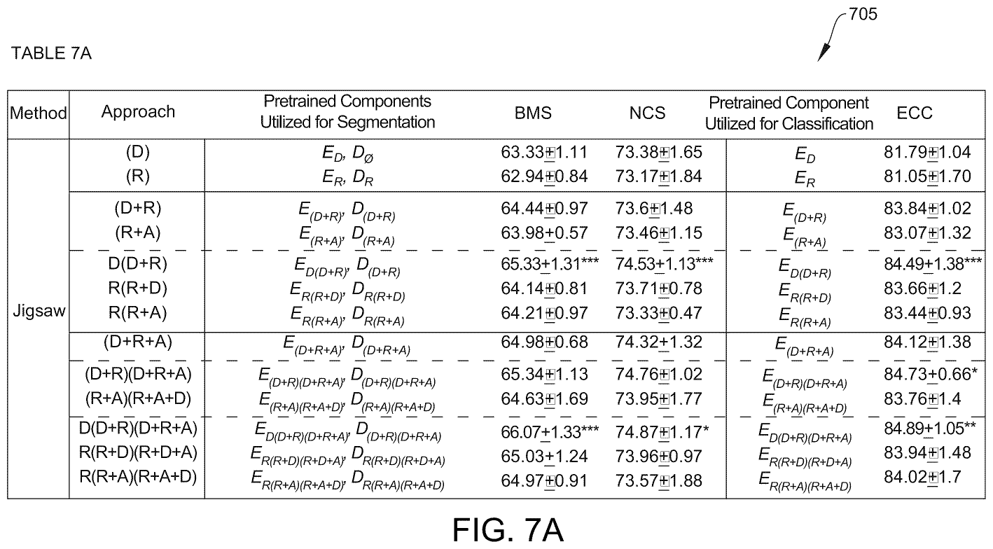

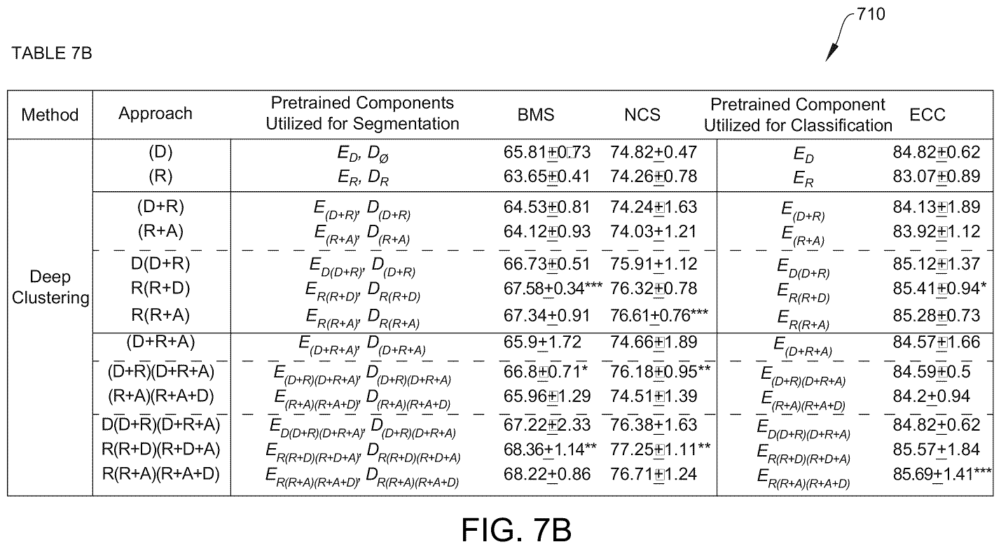

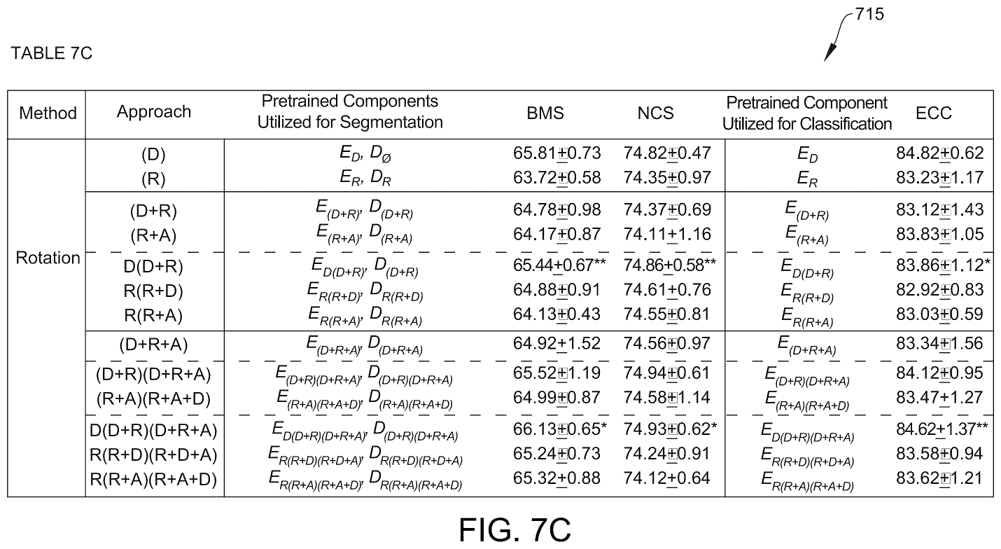

FIGS. 7A, 7B, and 7C depict Tables 7A, 7B, and 7C as set forth at elements 705, 710, and 715, showing a comparison of different training strategies. The mean and standard deviation (mean±s.d.) were reported based on ten trials, along with the statistic analysis (*p<0.5, **p<0.1, ***p<0.05) between the best (highest mean value) and the worst (lowest mean value) training strategies among similar setups (e.g., the same number of components and training steps) for Jigsaw, Deep Clustering, and Rotation. Increasing training steps generally improves performance. For Jigsaw and Rotation, starting with Discriminative Encoders yields better results, while for Deep Clustering, starting with reconstructive pre-training is more effective. This phenomenon is attributed to reconstructive pre-training enhancing feature learning for clustering.

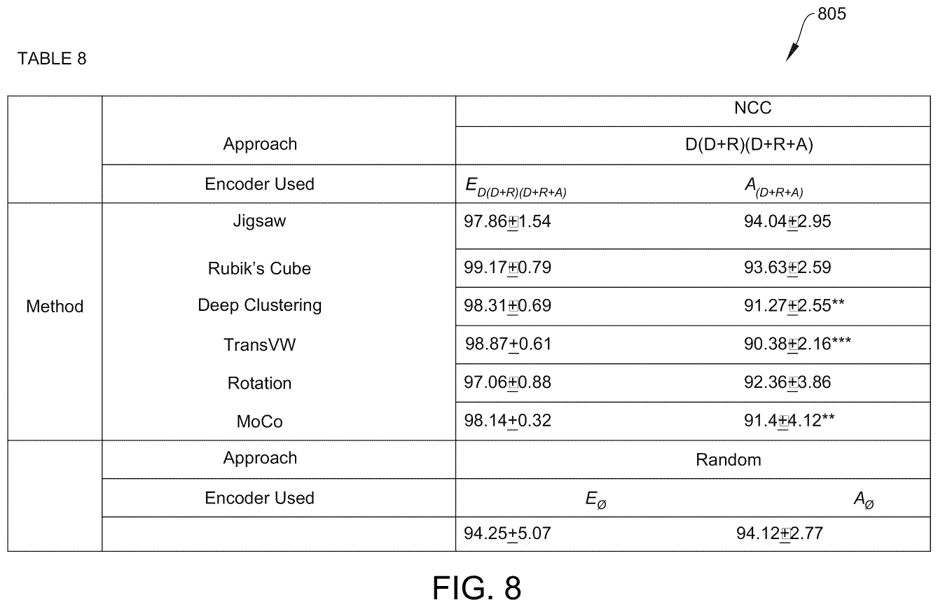

FIG. 8 depicts Table 8, as set forth at element 805, showing how adversarial encoders have learned weak representation, and that they are not suitable for transfer learning. The performance differences between Encoder E_{D(D+R)(D+R+A)} and Encoder A_{(D+R+A)} are easily observed; therefore, statistical analysis (*p<0.5, **p<0.1, ***p<0.05) was conducted between random initialization (A) and SSL initialized adversarial encoders (A_{(D+R+A)}). A_{(D+R+A)} pre-trained with all nine SSL methods perform worse than λ_{Ø}, indicating that their learned representations are not suitable for the target task. By contrast, SSL initialized discriminative encoders (E_{D(D+R)(D+R+A)}) all perform better than random initialization (E) for the target task.

### Comparing Different Incremental Pre-Training Strategies in the Unified Framework for Downstream Tasks:

The self-supervised methods that were selected are primarily discriminative/contrastive methods, with reconstructive and adversarial components being universal across all methods. It is possible to vary the reconstructive and adversarial components while maintaining the same discriminative/contrastive component across all the methods. However, this would introduce an exponentially larger number of combinations, which is beyond the scope of this work. When the only variable becomes the discriminative component, we further identify two types of discriminative methods: clustering or non-clustering. Performance of each of the methods were then tested through different training strategies.

The training strategies can also be identified as three types: starting training from the discriminative methods (SDM), start training from the reconstructive methods (SRM), and start training with combined methods (SCM). The SDM strategy includes D, D(D+R), and D(D+R)(D+R+A). The SRM strategy includes R, R(R+D), R(R+A), R(R+D) (R+D+A),
and R(R+A) (R+A+D). The SCM strategy includes (D+R), (D+R)(D+R+A), (R+A) (R+A+D), and (D+R+A). The combined use of discriminative and restorative methods (strategy D+R) consistently outperforms the individual use of either method (strategies D or R), as evident in Table 7. Furthermore, the models' performances are further enhanced with the pretraining of one of the methods (D or R). As shown in FIGS. 5A, 5B, 5C, 5D, 5E, and 5F, D(D+R) is always better than (D+R) across all target tasks with all nine methods and generally better than R(R+D) except for Deep Clustering and TransVW. Finally, pretraining was performed with all three components (D, R, and A) and observe that the stepwise incremental pretraining strategy consistently outperforms the combined training strategy, with the same number of training epochs being performed. Tables 7A, 7B, and 7C indicate that the D(D+R)(D+R+A) strategy performs best for Jigsaw and Rotation in downstream tasks, whereas R(R+D) (R+D+A) and R(R+A) (R+A+D) outperforms the D(D+R)(D+R+A) strategy for Deep Clustering. While incremental pre-training with SDM strategy typically yields the best performance for most methods, Deep Clustering benefits more from the incremental pre-training with SRM strategy. In experiments, it was concluded that for non-clustering types of discriminative methods, it is best practice to use the SDM strategy, while for clustering types of discriminative methods, SRM strategy yields the best performances. It is believed this phenomenon is due to the fact that reconstructive pretraining helps the encode learn features more consistent with appearance, thereby enabling clustering.

Adversarial encoders are not suitable for transfer learning as they learn weak representations: With stepwise incremental pretraining, two pretrained encoders are obtained, E_{D(D+R)(D+R+A)} and A_{D+R+A}, from the “United” model for target tasks. Their performance was evaluated on the task of lung nodule false positive reduction (NCC) with two settings: (1) linear evaluation, which fixes the pre-trained network and uses the features it computes to train a linear classifier for the target task, and (2) full fine-tuning of the pre-trained network for the target task. For linear evaluation, there is a significant performance difference between Encoder E_{D(D+R)(D+R+A)} and Encoder A_{D+R+A}. As shown in Table 8, the adversarial encoders are weaker than discriminative encoders. It is believed this is because the only pre-training supervision signal for the adversarial encoders is to distinguish real and fake images. This results in decreased performance for Jigsaw by AUC scores of 4.0%. Similarly, Rubik's Cube decreased by 6.7%, Deep Clustering by 7.2%, TransVW by 9.6%, Rotation by 3.9%, MoCo by 6.6%, BYOL by 6.0%, PCRL by 6.9%, and Swin UNETR by 5.1%. Furthermore, the adversarial encoders' performance is also worse than that of random initialized encoders A_{Ø}. This results in decreased performance for Jigsaw by AUC scores of 1.9%, for Rubik's Cube by 1%, for Deep Clustering by 2.8%, for TransVW by 5.7%, for Rotation by 2.3%, for MoCo by 3.6%, for BYOL by 3.3%, PCRL by 3.9%, and Swin UNETR by 2%. It is evident that the fixed features computed by the pretrained Encoder AD+R+A do not transfer well for the target task. Even when compared with the randomly initialized Encoder AØ, the computed features become less useful. The two encoders were further evaluated through full fine-tuning. While the Encoder A_{D+R+A} improves compared to its evaluation using linear evaluation, it still lags behind Encoder E_{D(D+R)(D+R+A)}. More importantly, the adversarial encoders' performance is not stable compared to discriminative encoders, as their standard deviations are higher.

In such a way, the specially developed and purpose-designed United framework implementation successfully integrates discriminative SSL methods with restorative and adversarial learning. The extensive experiments demonstrate that the pre-trained United models consistently outperform the SoTA baselines. This performance improvement is attributed to the stepwise incremental pre-training scheme, which not only stabilizes the pre-training but also unleashes the synergy of discriminative, restorative, and adversarial learning. It is expected that these pre-trained United models will exert an important impact on medical image analysis across diseases, organs, modalities, and specialties.

FIG. 9 sets forth Equations 1 through 5, which are referenced throughout the disclosure.

Thus, disclosed is a system comprising a memory to store instructions, and a processor to execute the instructions stored in the memory to perform the following operations: receiving at the system a training dataset comprising a plurality of medical images for training a unified artificial intelligence (AI) model; executing stepwise incremental pre-training operations to train the unified AI model, comprising: pre-training a discriminative encoder via discriminative learning, yielding a pre-trained discriminative encoder; attaching the pre-trained discriminative encoder to a restorative decoder to form a skip-connected encoder-decoder; pre-training the skip-connected encoder-decoder via joint discriminative and restorative learning, yielding the pre-trained discriminative encoder and a pre-trained restorative decoder; and associating the pre-trained skip-connected encoder-decoder with an adversarial encoder; finalizing training of the AI model by performing discriminative, restorative, and adversarial learning on the training dataset using the unified AI model, yielding the pre-trained discriminative encoder, the pre-trained restorative decoder, and a pre-trained adversarial encoder; and applying, via the unified AI model, each of discriminative, restorative, and adversarial learning operations through the discriminative encoder, the restorative decoder, and the adversarial encoder, generated via training of the unified AI model, for classifying and annotating medical images.

According to an embodiment, the system outputs a trained AI model for use with medical image analysis.

According to an embodiment, the system executing the stepwise incremental pre-training operations to train the unified AI model comprises performing self-supervised learning (SSL) training at each of the pre-training operations.

According to an embodiment, the performing self-supervised learning (SSL) training via 3D adapted SSL training operations including Rotation, Jigsaw, Rubik's Cube, Deep Clustering, TransVW, MoCo, BYOL, PCRL and Swin UNETR and each of the 3D adapted SSL training operations are augmented with supplemental 3D compatible components within the United framework for 3D medical imaging.

According to an embodiment, the system executing stepwise incremental pre-training operations to train the unified AI model comprises: training a discriminative encoder via discriminative learning; attaching the pre-trained discriminative encoder to a restorative decoder to form an encoder-decoder; training the encoder-decoder via combined discriminative and restorative learning; associating a pre-trained auto-encoder with the adversarial-encoder; and training the associated adversarial-encoder via full discriminative, restorative, and adversarial training.

According to an embodiment, executing stepwise incremental pre-training operations to train the unified AI model generates as an output a stable trained AI model.

Embodiments of the invention contemplate a machine or system within which embodiments may operate, be installed, integrated, or configured. In accordance with one embodiment, the system includes at least a processor and a memory therein to execute instructions including implementing any application code to perform any one or more of the methodologies discussed herein. Such a system may communicatively interface with and cooperatively execute with the benefit of remote systems, such as a user device sending instructions and data, a user device to receive output from the system.

A bus interfaces various components of the system amongst each other, with any other peripheral(s) of the system, and with external components such as external network elements, other machines, client devices, cloud computing services, etc. Communications may further include communicating with external devices via a network interface over a LAN, WAN, or the public Internet.

In alternative embodiments, the system may be connected (e.g., networked) to other machines in a Local Area Network (LAN), an intranet, an extranet, or the public Internet. The machine may operate in the capacity of a server or a client machine in a client-server network environment, as a peer machine in a peer-to-peer (or distributed) network environment, as a server or series of servers within an on-demand service environment. Certain embodiments of the machine may be in the form of a personal computer (PC), a tablet PC, a set top box (STB), a Personal Digital Assistant (PDA), a cellular telephone, a web appliance, a server, a network router, switch or bridge, computing system, or any machine capable of executing a set of instructions (sequential or otherwise) that specify and mandate the specifically configured actions to be taken by that machine pursuant to stored instructions. Further, the term “machine” shall also be taken to include any collection of machines (e.g., computers) that individually or jointly execute a set (or multiple sets) of instructions to perform any one or more of the methodologies discussed herein.

An exemplary computer system includes a processor, a main memory (e.g., read-only memory (ROM), flash memory, dynamic random access memory (DRAM) such as synchronous DRAM (SDRAM) or Rambus DRAM (RDRAM), etc., static memory such as flash memory, static random access memory (SRAM), volatile but high-data rate RAM, etc.), and a secondary memory (e.g., a persistent storage device including hard disk drives and a persistent database and/or a multi-tenant database implementation), which communicate with each other via a bus. Main memory includes code that implements the three branches of the SSL framework described herein, namely, the localizability branch, the composability branch, and the decomposability branch.

The processor represents one or more specialized and specifically configured processing devices such as a microprocessor, central processing unit, or the like. More particularly, the processor may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor may also be one or more special-purpose processing devices such as an application-specific integrated circuit (ASIC), a field programmable gate array (FPGA), a digital signal processor (DSP), network processor, or the like. The processor is configured to execute processing logic for performing the operations and functionality discussed herein.

The system may further include a network interface card. The system also may include a user interface (such as a video display unit, a liquid crystal display, etc.), an alphanumeric input device (e.g., a keyboard), a cursor control device (e.g., a mouse), and a signal generation device (e.g., an integrated speaker). According to an embodiment of the system, the user interface communicably interfaces with a user client device remote from the system and communicatively interfaces with the system via a public Internet.

The system may further include peripheral device (e.g., wireless or wired communication devices, memory devices, storage devices, audio processing devices, video processing devices, etc.).

A secondary memory may include a non-transitory machine-readable storage medium or a non-transitory computer readable storage medium or a non-transitory machine-accessible storage medium on which is stored one or more sets of instructions (e.g., software) embodying any one or more of the methodologies or functions described herein. The software may also reside, completely or at least partially, within the main memory and/or within the processor during execution thereof by the system, the main memory and the processor also constituting machine-readable storage media. The software may further be transmitted or received over a network via the network interface card.

In addition to various hardware components depicted in the figures and described herein, embodiments further include various operations which are described herein. The operations described in accordance with such embodiments may be performed by hardware components or may be embodied in machine-executable instructions, which may be used to cause a specialized and special-purpose processor having been programmed with the instructions to perform the operations described herein. Alternatively, the operations may be performed by a combination of hardware and software. In such a way, the embodiments of the invention provide a technical solution to a technical problem.

Embodiments also relate to an apparatus for performing the operations disclosed herein. This apparatus may be specially constructed for the required purposes, or it may be a special purpose computer selectively activated or reconfigured by a computer program stored in the computer. Such a computer program may be stored in a computer readable storage medium, such as, but not limited to, any type of disk including floppy disks, optical disks, CD-ROMs, and magnetic-optical disks, read-only memories (ROMs), random access memories (RAMs), EPROMs, EEPROMs, magnetic or optical cards, or any type of media suitable for storing electronic instructions, each coupled to a computer system bus.

While the algorithms and displays presented herein are not inherently related to any particular computer or other apparatus, they are specially configured and implemented via customized and specialized computing hardware which is specifically adapted to more effectively execute the novel algorithms and displays which are described in greater detail herein. Various customizable and special purpose systems may be utilized in conjunction with specially configured programs in accordance with the teachings herein, or it may prove convenient, in certain instances, to construct a more specialized apparatus to perform the required method steps. The required structure for a variety of these systems will appear as set forth in the description. In addition, embodiments are not described with reference to any particular programming language. It will be appreciated that a variety of programming languages may be used to implement the teachings of the embodiments as described herein.

Embodiments may be provided as a computer program product, or software, that may include a machine-readable medium having stored thereon instructions, which may be used to program a computer system (or other electronic devices) to perform a process according to the disclosed embodiments. A machine-readable medium includes any mechanism for storing or transmitting information in a form readable by a machine (e.g., a computer). For example, a machine-readable (e.g., computer-readable) medium includes a machine (e.g., a computer) readable storage medium (e.g., read only memory (“ROM”), random access memory (“RAM”), magnetic disk storage media, optical storage media, flash memory devices, etc.), a machine (e.g., computer) readable transmission medium (electrical, optical, acoustical), etc.

Any of the disclosed embodiments may be used alone or together with one another in any combination. Although various embodiments may have been partially motivated by deficiencies with conventional techniques and approaches, some of which are described or alluded to within the specification, the embodiments need not necessarily address or solve any of these deficiencies, but rather, may address only some of the deficiencies, address none of the deficiencies, or be directed toward different deficiencies and problems which are not directly discussed.

While the subject matter disclosed herein has been described by way of example and in terms of the specific embodiments, it is to be understood that the claimed embodiments are not limited to the explicitly enumerated embodiments disclosed. To the contrary, the disclosure is intended to cover various modifications and similar arrangements as are apparent to those skilled in the art. Therefore, the scope of the appended claims is to be accorded the broadest interpretation to encompass all such modifications and similar arrangements. It is to be understood that the above description is intended to be illustrative, and not restrictive. Many other embodiments will be apparent to those of skill in the art upon reading and understanding the above description. The scope of the disclosed subject matter is therefore to be determined in reference to the appended claims, along with the full scope of equivalents to which such claims are entitled.

Systems, Methods, and Apparatuses for Implementing Stepwise Incremental Pre-Training for Integrating Discriminative, Restorative, and Adversarial Learning into an AI Model

What is claimed is:

1. A system comprising:
a memory to store instructions;
a processor to execute the instructions stored in the memory to perform the following operations:
receiving at the system a training dataset comprising a plurality of medical images for training a unified artificial intelligence (AI) model;
executing stepwise incremental pre-training operations to train the unified AI model, comprising:
pre-training a discriminative encoder via discriminative learning, yielding a pre-trained discriminative encoder;
attaching the pre-trained discriminative encoder to a restorative decoder to form a skip-connected encoder-decoder;
pre-training the skip-connected encoder-decoder via joint discriminative and restorative learning, yielding the pre-trained discriminative encoder and a pre-trained restorative decoder; and
associating the pre-trained skip-connected encoder-decoder with an adversarial encoder;
finalizing training of the AI model by performing discriminative, restorative, and adversarial learning on the training dataset using the unified AI model, yielding the pre-trained discriminative encoder, the pre-trained restorative decoder, and a pre-trained adversarial encoder; and
applying, via the unified AI model, each of discriminative, restorative, and adversarial learning operations through the discriminative encoder, the restorative decoder, and the adversarial encoder, generated via training of the unified AI model, for classifying and annotating medical images.

2. The system of claim 1, further comprising:
outputting the trained AI model for use with medical image analysis.

3. The system of claim 1, wherein executing the stepwise incremental pre-training operations to train the unified AI model comprises performing self-supervised learning (SSL) training at each of the pre-training operations.

4. The system of claim 1:
wherein executing the stepwise incremental pre-training operations to train the unified AI model comprises performing self-supervised learning (SSL) training via 3D adapted SSL training operations including Rotation, Jigsaw, Rubik's Cube, Deep Clustering, TransVW, MoCo, BYOL, PCRL, and Swein UNETR; and
wherein each of the 3D adapted SSL training operations are augmented with supplemental 3D compatible components within the United framework for 3D medical imaging.

5. The system of claim 1, wherein executing stepwise incremental pre-training operations to train the unified AI model comprises:
training a discriminative encoder via discriminative learning;
attaching the pre-trained discriminative encoder to a restorative decoder to form an encoder-decoder;
training the encoder-decoder via combined discriminative and restorative learning; and
associating a pre-trained auto-encoder with the adversarial-encoder;
training the associated adversarial-encoder via full discriminative, restorative, and adversarial training.

6. The system of claim 1, wherein executing stepwise incremental pre-training operations to train the unified AI model generates as an output a stable trained AI model.

7. A computer-implemented method, performed by a system having at least a processor and a memory therein to execute instructions for implementing stepwise incremental pre-training for integrating discriminative, restorative, and adversarial learning into a single Artificial Intelligence (AI) model, comprising:
receiving at the system, a training dataset at the system comprising a plurality of medical images for training a unified AI model;
executing stepwise incremental pre-training operations to train the unified AI model, comprising:
pre-training a discriminative encoder via discriminative learning, yielding a pre-trained discriminative encoder;
attaching the pre-trained discriminative encoder to a restorative decoder to form a skip-connected encoder-decoder;
pre-training the skip-connected encoder-decoder via joint discriminative and restorative learning, yielding the pre-trained discriminative encoder and pre-trained restorative decoder; and;
associating the pre-trained skip-connected encoder-decoder with an adversarial encoder;
finalizing training of the AI model by performing discriminative, restorative, and adversarial learning on the training dataset using the unified AI model, yielding the pre-trained discriminative encoder, the pre-trained restorative decoder, and a pre-trained adversarial encoder; and
applying, via the unified AI model, each of discriminative, restorative, and adversarial learning operations through the discriminative encoder, the restorative decoder, and the adversarial encoder, generated via training of the unified AI model, for classifying and annotating medical images.

8. The computer-implemented method of claim 7, further comprising:
outputting the trained AI model for use with medical image analysis.

9. The computer-implemented method of claim 7, wherein executing the stepwise incremental pre-training operations to train the unified AI model comprises performing self-supervised learning (SSL) training at each of the pre-training operations.

10. The computer-implemented method of claim 7:
wherein executing the stepwise incremental pre-training operations to train the unified AI model comprises performing self-supervised learning (SSL) training via 3D adapted SSL training operations including Rotation, Jigsaw, Rubik's Cube, Deep Clustering, Trans VW, MoCo, BYOL, PCRL, and Swein UNETR; and
wherein each of the 3D adapted SSL training operations are augmented with supplemental 3D compatible components within the United framework for 3D medical imaging.

11. The computer-implemented method of claim 7, wherein executing stepwise incremental pre-training operations to train the unified AI model comprises:
training a discriminative encoder via discriminative learning;
attaching the pre-trained discriminative encoder to a restorative decoder to form an encoder-decoder;
training the encoder-decoder via combined discriminative and restorative learning; and
associating a pre-trained auto-encoder with the adversarial-encoder;
training the associated adversarial-encoder via full discriminative, restorative, and adversarial training.

12. The computer-implemented method of claim 7, wherein executing stepwise incremental pre-training operations to train the unified AI model generates as an output a stable trained AI model.

13. A non-transitory computer readable storage media having instructions stored thereupon that, when executed by a system having at least a processor and a memory therein, cause the processor to perform the following operations:
receiving at the system a training dataset comprising a plurality of medical images for training a unified Artificial Intelligence (AI) model;
executing stepwise incremental pre-training operations to train the unified AI model, comprising:
pre-training a discriminative encoder via discriminative learning, yielding a pre-trained discriminative encoder;
attaching the pre-trained discriminative encoder to a restorative decoder to form a skip-connected encoder-decoder;
pre-training the skip-connected encoder-decoder via joint discriminative and restorative learning, yielding the pre-trained discriminative encoder and a pre-trained restorative decoder; and;
associating the pre-trained skip-connected encoder-decoder with an adversarial encoder; and
finalizing training of the AI model by performing discriminative, restorative, and adversarial learning on the training dataset using the unified AI model, yielding the pre-trained discriminative encoder, the pre-trained restorative decoder, and a pre-trained adversarial encoder; and
applying, via the unified AI model, each of discriminative, restorative, and adversarial learning operations through the discriminative encoder, the restorative decoder, and the adversarial encoder, generated via training of the unified AI model, for classifying and annotating medical images.

14. The non-transitory computer readable storage media of claim 13, further comprising:
outputting the trained AI model for use with medical image analysis.

15. The non-transitory computer readable storage media of claim 13, wherein executing the stepwise incremental pre-training operations to train the unified AI model comprises performing self-supervised learning (SSL) training at each of the pre-training operations.

16. The non-transitory computer readable storage media of claim 13:
wherein executing the stepwise incremental pre-training operations to train the unified AI model comprises performing self-supervised learning (SSL) training via 3D adapted SSL training operations including Rotation, Jigsaw, Rubik's Cube, Deep Clustering, Trans VW, MoCo, BYOL, PCRL, and Swein UNETR; and
wherein each of the 3D adapted SSL training operations are augmented with supplemental 3D compatible components within the United framework for 3D medical imaging.

17. The non-transitory computer readable storage media of claim 13, wherein executing stepwise incremental pre-training operations to train the unified AI model comprises:
training a discriminative encoder via discriminative learning;
attaching the pre-trained discriminative encoder to a restorative decoder to form an encoder-decoder;
training the encoder-decoder via combined discriminative and restorative learning; and
associating a pre-trained auto-encoder with the adversarial-encoder;
training the associated adversarial-encoder via full discriminative, restorative, and adversarial training.

18. The non-transitory computer readable storage media of claim 13, wherein executing stepwise incremental pre-training operations to train the unified AI model generates as an output a stable trained AI model.
